(19) United States
(12) Patent Application Publication
(10) Pub. No.: US 2023/0407374 A1
(43) Pub. Date: Dec. 21, 2023

(54) SYSTEMS AND METHODS FOR HIGH THROUGHPUT SCREENING OF MOLECULAR INTERACTIONS

(71) Applicant: Children's Medical Center Corporation, Boston, MA (US)

(72) Inventors: Wesley Philip Wong, Cambridge, MA (US); Clinton H. Hansen, Brighton, MA (US)

(73) Assignee: Children's Medical Center Corporation, Boston, MA (US)

(21) Appl. No.: 18/035,835

(22) PCT Filed: Nov. 9, 2021
(86) PCT No.: PCT/US2021/058680

§ 371 Date: May 8, 2023

Related U.S. Application Data
(60) Provisional application No. 63/111,600, filed on Nov. 9, 2020.

(51) Int. Cl. C12Q 1/6818; C12Q 1/70

(52) U.S. Cl. CPC C12Q 1/6818; C12Q 1/70

(57) ABSTRACT

This disclosure provides new and improved systems and methods for analyzing binding interactions and for identifying and measuring agents that modulate such binding interactions, including weak binding interactions. The methods may be used in high throughput screening assays.

### RELATED APPLICATION

This application claims the benefit of U.S. Provisional Application No. 63/111,600 filed Nov. 9, 2020, the entire contents of which is incorporated by reference herein.

### GOVERNMENT LICENSE RIGHTS

This invention was made with government support under NIH NCI R21 Grant No. 80682 awarded by the National Institutes of Health. The Government has certain rights in the invention.

### BACKGROUND OF INVENTION

Molecular interactions, especially protein-protein interactions (PPIs), are an important group of potential drug targets (Arkin et al. 2014, Scott et al. 2016, Mabonga et al 2019). In vitro high-throughput screening (HTS) can be used to discover compounds that can increase or decrease the interaction between a pair of target molecules, often allosterically. Current successful in vitro HTS screening technologies are low-cost, homogeneous, no-wash assays that can be performed by robotic instruments in microplate format with an optical signal assayed on a plate reader. Current methods, however, face some limitations when assaying low-affinity interactions. Certain methods such as fluorescence polarization and time-resolved Forster resonance energy transfer (TR-FRET) require high concentrations of target (e.g., protein) approaching the respective affinity constants, which often can be in the micromolar range. Such high concentrations of targets (e.g., proteins) are unfeasible for HTS at least due to cost. Bead-based assays, such as Alphascreen, are limited by the high cost of beads, as well as the high concentration of target (e.g., protein) required to sufficiently coat the beads (e.g., tens to hundreds nM of target).

### SUMMARY OF INVENTION

High-throughput screening of molecular interactions, including protein-protein interactions, has been an integral part in discovering pharmaceutical compounds. Current techniques for interaction screening are limited in their ability to screen weak interactions (such as time-resolved FRET and fluorescence polarization) or are costly (such as Alphascreen). Presented herein are new and improved methodologies for screening of molecular interactions. These methodologies are referred to herein as linker-FRET and polymer-FRET, an example of which is nanoswitch-FRET. These are low-cost alternatives for screening molecular interactions, including but not limited to weak molecular interactions. Notably, both linker-FRET and polymer-FRET enable the assay of molecular interactions using binding partner concentrations (i.e., protein concentrations) that are significantly below the Kd of the interactions.

Generally, in linker-FRET, two target interacting partners are labeled with short (e.g., on the order of about 7-12 bp) FRET oligonucleotides, of which one or both have a linker (tether) between the partner and the oligonucleotide. Only when the two interacting partners interact, do the short FRET oligonucleotides bind to each other, resulting in a FRET signal.

In polymer-FRET, an example of which is nanoswitch-FRET, the formation of a loop brings FRET fluorophores in close proximity to each other, resulting in a FRET signal. The FRET fluorophores for can be incorporated onto oligonucleotides (e.g., short DNA sequences, on the order of about 7-12 base pairs) that only hybridize to each other when they are brought in close proximity as the loop closes. The zipper so created serves two purposes: it positions the fluorophores relative to each other for maximum FRET efficiency and also enhances the affinity of weak target interactions. Multiple interacting partners can also be included for an avidity effect to assay very weak target interactions.

These methodologies have been demonstrated using weak DNA-DNA oligonucleotide interactions as an example, resulting in a suitable assay for interactions on the order of 15 μM for nanoswitch-FRET and 75 μM for linker-FRET.

An additional variations on linker-FRET and nanoswitch-FRET are provided herein including single fluorophore linker-FRET used together with fluorescence polarization.

Accordingly, one aspect of this disclosure provides a system for analyzing modulation of a binding interaction, comprising a first member of a binding pair, conjugated to a first oligonucleotide, wherein the first oligonucleotide is conjugated to a FRET donor fluorophore, and a second member of the binding pair, conjugated to a second oligonucleotide, wherein the second oligonucleotide is conjugated to a FRET acceptor fluorophore, wherein the first and second members bind to each other with a member dissociation constant, Kd, in the range of about 100 pM to about 100 μM, and wherein the first and second oligonucleotides hybridize to each other with an oligonucleotide dissociation constant, Kd, that ranges from about 100 pM to about 1 μM.

Various embodiments apply to various aspects provided herein. These various embodiments are recited once for simplicity and brevity. They are as follows:

The first and second oligonucleotides may be less than 30 base pairs, less than 20 base pairs, or less than 15 base pairs. In some embodiments, they are about 5-20 base pairs in length, or about 5-15 base pairs in length, or about 5-10 base pairs in length, including 9 base pairs in length. The length may also be in the range of 7 to 12 base pairs.

In some embodiments, the first member is conjugated to the first oligonucleotide using a first linker. In some embodiments, the second member is conjugated to the second oligonucleotide using a second linker. In some embodiments, the first and/or the second linker has a length of about 2-14 nm. In some embodiments, the first and/or the second linker comprises 1-6 adjacent 18-atom hexa-ethyleneglycol spacers. In some embodiments, the first and/or the second linker comprises four adjacent 18-atom hexa-ethyleneglycol spacers.

In some embodiments, the first member of the binding pair is a virus or viral component capable of binding to a mammalian cell and the second member of the binding pair is a mammalian cell or a component of the mammalian cell to which the viral component binds. In some embodiments, the virus is Sars-CoV-2 or the viral component is Sars-CoV-2 receptor binding domain (RBD) and the mammalian cell is a human cell or the component of the mammalian cells is receptor ACE2. In some embodiments, the member Kd is about 400 nM and the oligonucleotide Kd is about 50 nM.

In some embodiments, once the first and second oligonucleotides are hybridized to each other, the donor and acceptor FRET fluorophores are spaced apart by 1 to 8 base pairs, optionally 4-7 base pairs, further optionally 4 or 5 base pairs. In some embodiments, the donor FRET fluorophore is Alexa 555 and the acceptor FRET fluorophore is Alexa 647, or wherein the donor FRET fluorophore is Alexa 488 and the acceptor FRET fluorophore is Alexa 555, or wherein the donor FRET fluorophore is Alexa 594 and the acceptor FRET fluorophore is Alexa 647.

In some embodiments, the first member and/or second member is a protein.

Another aspect of this disclosure provides a method for detecting an agent capable of modulating a binding interaction, comprising (a) providing (i) a first member of a binding pair, conjugated to a first oligonucleotide, wherein the first oligonucleotide is conjugated to a FRET donor fluorophore, and (ii) a second member of the binding pair, conjugated to a second oligonucleotide, wherein the second oligonucleotide is conjugated to a FRET acceptor fluorophore, wherein the first and second members bind to each other with a member dissociation constant. Kd, in the range of about 100 pM nM to about 100 μM, and wherein the first and second oligonucleotides hybridize to each other with an oligonucleotide dissociation constant, Kd, that ranges from about 100 pM to about 1 μM and is comparable to or greater than the concentration of oligonucleotides used in the assay, (b) combining the first member and the second member with a sample, under conditions that allow binding of the first member to the second member and that allow binding of the first oligonucleotide to the second oligonucleotide, optionally wherein the first member and second member are provided in concentrations ranging from about 100 pM to about 100 nM, (c) measuring test fluorescence from the FRET acceptor, and (d) (1) identifying the sample as containing an agent that (i) decreases binding of the first member to the second member as a test fluorescence that is less than a control fluorescence measured after combining the first member and the second member under the same conditions as (b) but in the absence of the sample, or (ii) increases binding of the first member to the second member as a test fluorescence that is greater than a control fluorescence measured after combining the first member and the second member under the same conditions as (b) but in the absence of the sample; or (2) identifying the sample as containing an agent that modulates binding of the first member to the second member, wherein a test fluorescence that is less than a control fluorescence, measured in the absence of the sample, is indicative of an agent that decreases binding of the first member to the second member, and a test fluorescence that is greater than a control fluorescence, measured in the absence of the sample, is indicative of an agent that increases binding of the first member to the second member.

In some embodiments, the oligonucleotide Kd is about 1 times, 2 times, 5 times, 10 times or higher than the concentration of oligonucleotides used in the assay.

In some embodiments, the first member is conjugated to the first oligonucleotide using a first linker. In some embodiments, the second member is conjugated to the second oligonucleotide using a second linker. In some embodiments, the first and/or the second linker has a length of about 2-14 nm. In some embodiments, the first and/or the second linker comprises 1-6 adjacent 18-atom hexa-ethyleneglycol spacers. In some embodiments, the first and/or the second linker comprises four adjacent 18-atom hexa-ethyleneglycol spacers.

In some embodiments, the method is a method that detects an agent that decreases binding of the first member to the second member. In some embodiments, the method is a method that detects an agent that increases binding of the first member to the second member.

In some embodiments, the sample is a blood or serum sample, optionally a blood or serum sample from a human subject, further optionally a human serum sample.

In some embodiments, the first member of the binding pair is a virus or viral component capable of binding to a mammalian cell and the second member of the binding pair is a mammalian cell or a component of the mammalian cell to which the viral component binds. In some embodiments, the virus is Sars-CoV-2 or the viral component is Sars-CoV-2 receptor binding domain (RBD) and the mammalian cell is a human cell or the component of the mammalian cells is receptor ACE2. In some embodiments, the member Kd is about 400 nM and the oligonucleotide Kd is about 50 nM.

In some embodiments, the method is a method that detects an agent that decreases binding of the first member to the second member and the agent is an antibody, and the sample is a blood or serum sample.

In some embodiments, once the first and second oligonucleotides are hybridized to each other, the donor and acceptor FRET fluorophores are spaced apart by 1 to 8 base pairs, optionally 4-7 base pairs, further optionally 4 or 5 base pairs. In some embodiments, the donor FRET fluorophore is Alexa 555 and the acceptor FRET fluorophore is Alexa 647, or wherein the donor FRET fluorophore is Alexa 488 and the acceptor FRET fluorophore is Alexa 555, or wherein the donor FRET fluorophore is Alexa 594 and the acceptor FRET fluorophore is Alexa 647.

In some embodiments, the first and second members are provided at concentrations lower than the member Kd, optionally 10-fold lower, 50-fold lower, 100-fold lower, 500-fold lower, 1,000-fold lower, or 5,000 lower.

In some embodiments, the first member and second member and sample are combined at about room temperature for about 30 minutes. In some embodiments, the method is carried out in a single well of a multiwell plate, optionally wherein the multiwell plate is a 384-well plate.

In some embodiments, the first member and/or second member is a protein.

In some embodiments, the method does not comprise a washing step between steps (b) and (c).

In some embodiments, the first and second member are in free-form in solution.

In some embodiments, the first and second members are attached to a single polymer, with sufficient distance between the first and second members to allow binding of the first and second members to each other.

Another aspect of this disclosure provides a system for analyzing modulation of a binding interaction, comprising a first member of a binding pair, conjugated to a first oligonucleotide, wherein the first oligonucleotide is conjugated to a fluorophore, and

a second member of the binding pair, conjugated to a second oligonucleotide,

wherein the first and second members bind to each other with a member dissociation constant, Kd, in the range of about 100 pM nM to 100 μM and wherein the first and second oligonucleotides hybridize to each other with an oligonucleotide dissociation constant, Kd, that ranges from about 100 pM to about 1 μM.

In some embodiments, the first member is conjugated to the first oligonucleotide using a linker. In some embodiments, the linker has a length of about 2-14 nm. In some embodiments, the linker comprises 1-6 adjacent 18-atom hexa-ethyleneglycol spacers. In some embodiments, the first linker comprises four adjacent 18-atom hexa-ethyleneglycol spacers.

Another aspect of this disclosure provides a method for detecting an agent capable of modulating a binding interaction, comprising (a) providing (i) a first member of a binding pair, conjugated to a first oligonucleotide, wherein the first oligonucleotide is conjugated to a fluorophore, and (ii) a second member of the binding pair, conjugated to a second oligonucleotide, wherein the first and second members bind to each other with a member dissociation constant, Kd, in the range of about 100 pM to about 100 μM, and wherein the first and second oligonucleotides hybridize to each other with an oligonucleotide dissociation constant, Kd, that ranges from about 100 pM to about 1 μM and is about equal to or greater than the concentration of oligonucleotides used in the assay, optionally wherein said concentrations are about 100 pM to about 100 nM, (b) combining the first member and the second member with a sample, under conditions that allow binding of the first member to the second member and that allow binding of the first oligonucleotide to the second oligonucleotide, (c) measuring test fluorescence using fluorescence polarization, and (d) identifying the sample as containing an agent that (i) decreases binding of the first member to the second member as a test fluorescence that is less than a control fluorescence measured after combining the first member and the second member under the same conditions as (b) but in the absence of the sample, or (ii) increases binding of the first member to the second member as a test fluorescence that is greater than a control fluorescence measured after combining the first member and the second member under the same conditions as (b) but in the absence of the sample.

Another aspect of this disclosure provides a system for analyzing modulation of a binding interaction, comprising a polymer conjugated to (i) a first member of a binding pair and a second member of a binding pair, wherein the first and second members bind to each other with a member dissociation constant, Kd, of 100 pM to 100 μM, and wherein the polymer adopts a looped conformation when the first and second member bind to each other, and (ii) a first FRET oligonucleotide conjugated to a first FRET fluorophore and a second FRET oligonucleotide conjugated to a second FRET fluorophore, wherein the first and second FRET oligonucleotides hybridize to each other with a FRET oligonucleotide dissociation constant, Kd, and wherein one of the FRET fluorophores is a FRET donor fluorophore and the other of the FRET fluorophores is a FRET acceptor fluorophore.

In some embodiments, the polymer is a nucleic acid, a linear amino acid chain, or polyethylene glycol.

Another aspect of this disclosure provides a system for analyzing modulation of a binding interaction, comprising a complex comprising a single-stranded scaffold nucleic acid hybridized to a plurality of single-stranded oligonucleotides thereby forming a linear partially double-stranded nucleic acid, wherein a first single-stranded oligonucleotide in the plurality is linked to a first member of a binding pair, and a second single-stranded oligonucleotide in the plurality is linked to a second member of a binding pair, wherein the first and second members bind to each other with a member dissociation constant, Kd, of 100 pM to 100 μM, and wherein the complex adopts a looped conformation when the first and second member bind to each other, wherein a first FRET oligonucleotide conjugated to a first FRET fluorophore is conjugated to the complex and a second FRET oligonucleotide conjugated to a second FRET fluorophore is conjugated to the complex, wherein the first and second FRET oligonucleotides hybridize to each other with a FRET oligonucleotide dissociation constant, Kd, optionally in the range of about 100 pM to 1 μM, and wherein one of the FRET fluorophores is a FRET donor fluorophore and the other of the FRET fluorophores is a FRET acceptor fluorophore.

In some embodiments, the first FRET oligonucleotide is conjugated to the complex using a first linker and/or the second FRET oligonucleotide is conjugated to the complex using a second linker, optionally wherein the first and/or second linker comprises one or more 18-atom hexa-ethyleneglycol spacers.

In some embodiments, the oligonucleotides are positioned along the length of the complex in order, optionally as first oligonucleotide, first FRET oligonucleotide, second FRET oligonucleotide, and second oligonucleotide.

In some embodiments, the first and second FRET oligonucleotides bind to each other only upon binding of the first and second members to each other.

In some embodiments, the first oligonucleotide and the first FRET oligonucleotide are about 30 base pairs apart. In some embodiments, the second oligonucleotide and the second FRET oligonucleotide are about 30 base pairs apart.

In some embodiments, a third and a fourth oligonucleotide, flank the first and second oligonucleotides respectively, comprise a first member and a second member respectively.

Another aspect of this disclosure provides a method for detecting an agent capable of modulating a binding interaction, comprising (a) combining a polymer conjugated to a first member of a binding pair and a second member of a binding pair, as described above, with a sample, under conditions that allow binding of the first member to the second member and that allow binding of the first FRET oligonucleotide to the second FRET oligonucleotide, (b) measuring test fluorescence from the FRET acceptor, and (c) identifying the sample as containing an agent that (i) decreases binding of the first member to the second member as a test fluorescence that is less than a control fluorescence measured after incubating the polymer under the same conditions as (a) in the absence of the sample, or (ii) increases binding of the first member to the second member as a test fluorescence that is greater than a control fluorescence measured after incubating the polymer under the same conditions as (a) in the absence of the sample.

Another aspect of this disclosure provides a method for detecting an agent capable of modulating a binding interaction, comprising (a) combining the complex comprising a single-stranded scaffold nucleic acid hybridized to a plurality of single-stranded oligonucleotides, two of which are conjugated to a first member and second member, and optionally two more of which are conjugated to FRET oligonucleotides (or are FRET oligonucleotides with 5′ and 3′ overhangs), with a sample, under conditions that allow binding of the first member to the second member and that allow binding of the first FRET oligonucleotide to the second FRET oligonucleotide, (b) measuring test fluorescence from the FRET acceptor, and (c) identifying the sample as containing an agent that (i) decreases binding of the first member to the second member as a test fluorescence that is less than a control fluorescence measured after incubating the complex under the same conditions as (a) in the absence of the sample, or (ii) increases binding of the first member to the second member as a test fluorescence that is greater than a control fluorescence measured after incubating the complex under the same conditions as (a) in the absence of the sample.

These and other aspects and embodiments will be described in greater detail herein.

### BRIEF DESCRIPTION OF DRAWINGS

FIGS. 1A-1B show schematics of linker-FRET for target (binding partner, member, as such terms are used interchangeably herein) interaction screening.

FIG. 2 shows FRET ratio as a function of fluorophore spacing along the length of hybridized FRET oligonucleotides.

FIGS. 3A-3B show data from a linker-FRET assay.

FIGS. 4A-4B show data from a linker-FRET assay for ACE2 and RBD.

FIG. 5 shows a schematic of linker-FP for target interaction screening.

FIGS. 6A-6B show schematics of a preparation procedure for linker-FRET with proteins as binding partners, including suitable but non-limiting chemistries and labeling positions.

FIGS. 7A-7D show various DNA nanoswitch configurations and readouts.

FIGS. 8A-8C show schematics of various nanoswitch-FRET configurations.

FIGS. 9A-9B show bar graphs providing FRET ratio as a function of FRET oligonucleotide (also called detection oligonucleotide) affinity.

FIG. 10 shows dose response curves for a target interaction sequence.

FIG. 11 shows the Z-prime value of a nanoswitch-FRET assay, indicative of assay quality.

### REFERENCE TO A SEQUENCE LISTING SUBMITTED AS A TEXT FILE

This application contains a Sequence Listing which has been submitted in ASCII format and is hereby incorporated by reference in its entirety. Said ASCII copy, created on Nov. 9, 2021, is named C123370198WO00-SEQ-MAT and is 7,085 bytes in size.

### DETAILED DESCRIPTION OF INVENTION

Aspects of this disclosure provide compositions and methods of use relating to linker-FRET and polymer-FRET, one example of the latter being nanoswitch-FRET. Linker-FRET and polymer-FRET, including nanoswitch FRET, may be used to a number of applications. One important application is analysis of one or more binding interactions, and specifically analyzing modulation of a binding interaction. Each of these will be described in greater herein.

Linker-FRET and polymer-FRET, including nanoswitch FRET, may be used to a number of applications. One important application is analysis of one or more binding interactions, and specifically analyzing modulation of a binding interaction.

Binding interactions that may be studied are defined as non-covalent interactions that occur between two moieties. Typically, these moieties are known, and there are referred to as first and second members of a binding partner pair. One or both members may be amino acid in nature, such as proteins or peptides. In some embodiments, both members are amino acid in nature, optionally both are proteins. Other moieties may be nucleic acids, carbohydrates, lipids, and the like. Examples of binding pairs include ligand and receptor pairs, pathogen and target pairs, transcription factor and nucleic acid pairs, and the like.

As will be described below in greater detail, aspects of linker-FRET and polymer-FRET, such as nanoswitch FRET, are directed to binding interactions having a range of binding affinities. As will be understood in the art, binding affinity is commonly measured and thus expressed in terms of equilibrium dissociation constant, or Kd. Kd refers to the concentration at which half of the binding partners are bound to each other. Higher values of Kd represent weaker binding interactions, and lower values of Kd represent stronger binding interactions.

The methods provided herein may be used to analyze and modulate binding interactions having Kd in the range of about 100 pM to about 100 μM, including Kd in the range of about 1 nM to about 100 μM, including Kd in the range of about 100 nM to about 100 μM, including Kd in the range of about 1 to about 100 μM, including Kd in the range of about 10 to about 100 μM. Some binding interactions that can be analyzed using the methods provided herein have a Kd in the range of about 50 nM to 1 μM, or about 100 nM to about 800 nM, or about 100 nM to about 600 nM, or about 200 nM to about 500 nM, or about 300 nM to about 500 nM, or about 400 nM.

In some embodiments, the binding interactions being analyzed are considered weak binding interactions. In some embodiments, the binding interactions being analyzed are considered intermediate binding interactions. In still other embodiments, the binding interactions being analyzed are considered strong binding interactions.

Modulation of a binding interaction, as used herein, refers to a change in a binding interaction, such as an increase or a decrease of a binding interaction. The methods generally involve detecting and optionally measuring an interaction between two members of a binding pair in the absence (control) or presence (test) of a putative agent that impacts the binding interaction. Agents that decrease the binding interaction may be referred to as inhibitors or antagonists. Agents that increase the binding interaction may be referred to as enhancers or agonists.

Changes in binding interactions (i.e., decreases or increases) are measured as changes in fluorescence resonance energy transfer, or FRET, as will be described below. An inhibitor or antagonist may cause a decrease in FRET signal that is on the order of about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, or more of the FRET signal that is measured in the absence of the inhibitor or antagonist (i.e., the FRET signal measured in the control assay). It is to be understood that, as provided herein, an inhibitor or antagonist that causes a decrease in FRET on the order to about 10% may causes at least that level of decrease, but it may cause a greater decrease. An example of such an inhibitor may be an antibody, such as a neutralizing antibody.

Methods directed to detecting an inhibitor or antagonist or measuring inhibition of binding can be used in clinical applications, such as those directed to detecting or measuring the level of an inhibitor or antagonist in a patient sample. One such example discussed in greater detail below is detection of anti-Sars-CoV-2 antibodies in a subject. In this context, the assay can be used to determine whether a subject has immunity to the virus, whether the subject has responded to a vaccine, and how long the subject's immunity has lasted either post-infection or post-vaccination. The methods provided herein are suitable for high-throughput screening (or assay) and thus amenable to large-scale analyses such as those required in monitoring immunity in a pandemics, such as the Covid pandemic. Those of skill will however appreciate that the methods may be useful in other settings as well.

An enhancer or agonist may cause an increase in FRET signal that is on the order of about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, or more of the FRET signal that is measured in the absence of the enhancer or agonist (i.e., the FRET signal measured in the control assay). It is to be understood that, as provided herein, an enhancer or agonist that causes an increase in FRET on the order to about 10% may causes at least that level of increase, but it may cause a greater increase. Some aspects of this disclosure may be alternatively used to analyze dose-response curves including to analyze, and thereby determine, the amount of an inhibitor that is required to achieve a desired decrease in binding interaction or to analyze, and thereby determine, the amount of an enhancer that is required to achieve a desired increase in binding interaction.

Förster (or fluorescence) resonance energy transfer (FRET) is a method for analyzing interaction and proximity of two moieties by labeling one moiety with a FRET donor moiety and the other moiety with a FRET acceptor moiety. The donor fluorophore absorbs energy of a certain wavelength and emits energy at a different wavelength. The donor fluorophore's emission is then absorbed by the FRET acceptor fluorophore which is then emits at yet a different wavelength. The ultimate FRET signal is the emission from the acceptor fluorophore. The transfer of energy from the donor to the acceptor only occurs, however, if the donor and acceptor are in sufficiently close proximity. Thus, in the context of this disclosure, the acceptor emission fluorescence is only detected if the FRET oligonucleotides are hybridized to each other, and this in turn occurs when the members of the binding pair are bound to each other.

Various FRET donor/acceptor pairs are known in the art and would be suitable for use in the aspects of this disclosure. Non-limiting donor-acceptor pair examples include Alexa 555/Alexa 647, Alexa 488/Alexa 555, Alexa 594/Alexa 647, and Cy3/Cy5.

Each FRET pair fluorophore can be attached to an oligonucleotide, referred to as a FRET oligonucleotide. These FRET oligonucleotides can hybridize to each other, and once hybridized, the FRET donor and acceptor fluorophores are brought into close enough proximity to effect the energy transfer from the donor to the acceptor. The FRET donor and acceptor may be spaced apart by about 1 to 8 base pairs (bps), or about 4 to 7 bps, or about 4-5 bps. In some embodiments, the FRET donor and acceptor are spaced apart by 4 or 5 bps. Appropriate spacing between the donor and acceptor, to ensure that maximum energy transfer occurs, may be determined by analyzing the ratio of the fluorescence signals detected at the characteristic emission wavelengths of the acceptor and the donor. The higher the ratio, the more efficient the energy transfer and the more preferred the distance between the donor and acceptor. The Examples demonstrate how such a measurement is performed.

A number of exemplifications provided herein used FRET (and corresponding FRET donor and acceptor fluorophores). However, other fluorophore pairings are also contemplated such as a split fluorophore that only yields a signal when two parts are brought together, or a dye that substantially increases in fluorescence when it binds to double stranded DNA, such as SYBR gold.

The assays disclosed herein were demonstrated without the use of any specialized fluorophores, such as those required for TR-FRET. Nevertheless, fluorophores for TR-FRET could be incorporated instead to reduce the background and improve Z′.

Additionally, a built-in monochromator-based system can be used, as can a filter set that can read both the FRET and non-FRET emission from the same excitation in order to reduce noise.

This disclosure refers to various components being conjugated to one another. Such conjugation may be covalent or it may non-covalent unless specifically indicated.

Further, conjugation may be direct or indirect conjugation. Direct conjugation intends that a first moiety is itself conjugated to a second moiety without any other moiety in between. An indirect conjugation intends that a first moiety is conjugated to second moiety, through an intermediate such as a linker (which is alternatively known as a spacer). Suitable linkers also will not interfere with the binding interaction of the oligonucleotides to each other or the binding pair members to each other. For example, the linkers will not adopt a secondary structure and will not be self-repulsive. An example of a suitable linker is a hexa-ethylene-glycol, such as an 18-atom hexa-ethylene glycol. Other potential linkers include PEG, including heterobifunctional PEG with the number PEG subunits ranging from 4 to 36, with one end designed to attach to the oligonucleotide and one end designed to attach to the binding partner, such as Azido-PEG24-NHS ester. Still other linkers could be nucleic acids. Linkers are known in the art and a person of skill in the art would readily identify suitable linkers based on the disclosure herein.

A variety of embodiments provided herein involve proteins conjugated to nucleic acids such as oligonucleotides. For example, in nanoswitch-FRET, binding partners such as proteins are bound to tile oligonucleotides. This can be accomplished in a number of ways. For example, free amines on a protein could be reacted with a bifunctional NHS-peg4-DBCO, and then reacted with an azide-modified oligonucleotide. A NHS-peg4-tetrazine linker can also be used to react with a TCO-modified oligonucleotide. Reactive groups, other than free amines, could also be used, such as but not limited to free cysteines reacting with maleimide chemistry, or a SNAP-tag reacting with a benzylguanine derivative. The coupled proteins can then be purified from unbound oligonucleotide using methods including, but not limited to, FPLC, precipitation, or a pulldown using a specific tag present on the proteins, such as a His- or Flag-tag. A similar methodology can be used in linker-FRET or linker-FP.

This disclosure contemplates and embraces a variety of conjugation methodologies for conjugating members to linker or to oligonucleotides, or to conjugate linkers to oligonucleotides, and the like.

In some embodiments, suitable linkers will be of sufficient length, as described herein, to allow oligonucleotides, such as FRET oligonucleotides, to bind to each other upon binding of members of the binding pair. The linkers may be on the same length, or may be of different lengths depending on the embodiment. In some embodiments, the linker length may be adjusted by using one or more linker units, arranged adjacently. The number of linker units may vary from 1-10, including 1-6, 2-8 and 4-6 units, depending on the embodiment. Some embodiments use 1-6 units, others use 4 units. Linker units here can refer to number of units or monomers of a polymer, such as PEG or nucleic acid. Regardless of composition, linkers may have a variety of lengths depending on the embodiment. In some embodiments, a length in the range of about 2 to about 25 nm, or about 2 to about 20 nm, or about 2 to about 15 nm, or about 2 to about 10 nm, or about 2 to about 5 nm. In some embodiments, the length is the in range of about 2 to about 14 nm, or is about 2, about 3, about 4, about 5, about 6, about 7, about 8, about 9, about 10, about 11, about 12, about 13, or about 14 nm.

One aspect of linker-FRET is directed to systems for analyzing modulation of a binding interaction. Systems provided herein refer to the collection of components to be used in analyzing binding interactions. Such systems may further comprise instructions for using the recite components to analyze binding interactions, including without limitation the concentrations of components to be used when performing binding interaction assays.

Thus, one aspect of this disclosure provides a system for analyzing modulation of a binding interaction comprising a first and a second member of a binding pair. The first member is conjugated to a first oligonucleotide, and the second member is conjugated to a second oligonucleotide. The oligonucleotides each comprise a fluorophore and the two fluorophores comprise a FRET donor/acceptor pair. Thus, the first oligonucleotide may be conjugated to a FRET donor fluorophore and the second oligonucleotide may be conjugated to a FRET acceptor fluorophore, or vice versa. Oligonucleotides comprising a donor or an acceptor FRET fluorophore may be referred to herein as FRET oligonucleotides.

The conjugation of the first and second members to the first and second oligonucleotides (e.g., FRET oligonucleotides) is typically covalent. It is also typically an indirect conjugation and involves a linker positioned between the member and the oligonucleotide, such as but not limited to a hexa-ethylene glycol linker (e.g., one or more 18-atom hexa-ethylene glycol units). The first member may be conjugated to the first oligonucleotide via a first linker, and the second member may be conjugated to the second oligonucleotide via a second linker. The first and second linkers may be identical in length and composition or they may differ from each other in length and/or composition.

As noted herein, in some embodiments, the first and second members bind to each other with a member dissociation constant, Kd, in the range of about 100 pM to about 100 μM. In other embodiments, the member Kd may be in the range of about 1 nM to about 100 μM. The oligonucleotides hybridize to each other with an oligonucleotide dissociation constant, Kd. The oligonucleotide Kd may range from about 100 pM to about 1 μM or about 1 nM to about 1 μM, and significantly is comparable to (about equal to) or greater than the concentration of the oligonucleotides used in the assay. The assays may use components, such as the member-oligonucleotide conjugates at concentrations of about 100 pM to about 100 nM, in some embodiments. Accordingly, if the oligonucleotide Kd is about 1 nM, then each of the oligonucleotides are used at a concentration of about equal to or less than 1 nM in the assay, and accordingly the concentration of each of the member-oligonucleotide conjugates is also equal to or less than 1 nM in the assay since the oligonucleotides are provided in the conjugate (and not free) form. In some embodiments, the oligonucleotide Kd is about 1 times, 2 times, 5 times, 10 times or higher than the concentration of each oligonucleotides used in the assay. As used herein, and unless otherwise indicated, the concentration of the oligonucleotide used in an assay is also the concentration of a member used in an assay, when the two are conjugated to each other. The oligonucleotide length and sequence composition may be chosen and/or designed by a person skilled in the art based on these criteria.

In some embodiments of the various aspects provided herein, the first and second members bind to each other with a member dissociation constant, Kd, in the range of about 100 μM to 100 M, and the first and second oligonucleotides hybridize to each other with an oligonucleotide dissociation constant, Kd, that ranges from about 100 pM to about 1 μM and is comparable to (about equal to) or greater than the concentration of oligonucleotides (or member-oligonucleotide conjugates) used in the methods provided herein, including but not limited to about 100 pM to about 100 nM.

The use of member-oligonucleotide conjugates as described herein creates a system in which the members (i.e., binding partners) and oligonucleotides have a lower Kd as compared to their respective Kd when each is in an unconjugated form. For example, a first and second member may have a Kd of 100 pM when both are present in free-form and unconjugated to other interacting components, such as the FRET oligonucleotides. However, the same first and second members may have a lower Kd, for example on the order to about 100 nM, when each is conjugated to an interacting component such as a FRET oligonucleotide. Thus, the Kd of a member in a member-oligonucleotide conjugate is lower than the Kd of the member in an unconjugated form. The ability to reduce the member Kd by conjugating it to another entity that stabilizes the member-member binding interaction means that lower amounts (or concentrations) of members are needed to perform the assays, thereby making such assays more cost-effective.

In other embodiments, the oligonucleotide Kd may be above or below the member Kd.

Thus, as will be apparent, there is a relationship between the member Kd, the oligonucleotide Kd, and the concentration of the components used in the assay. Typically, the components (i.e., the members and the oligonucleotides) will be conjugated to each other (e.g., the first member will be conjugated to the first oligonucleotide, and the second member will be conjugated to the second oligonucleotide) and therefore the concentration of a first member, a first oligonucleotide and a first member-oligonucleotide conjugate will be the same. Similarly, the concentration of a second member, a second oligonucleotide and a second member-oligonucleotide conjugate will be the same. Various factors must be taken into consideration when designing an appropriate assay. In some assays (and thus some systems), the member binding affinity will be low (or weak) and it will benefit from the hybridization of the oligonucleotides in order to provide stability and thus detection of the member binding interaction. However, the Kd of the uncoupled oligonucleotides (to each other) should be higher than the concentration at which the coupled member-oligonucleotides are used in the assay, in order to ensure that the binding interaction being monitored and measured is actually the member binding interaction and not the oligonucleotide binding interaction. In other words, the oligonucleotide binding interaction is intended to occur only once the first and second members are sufficiently close and should serve to stabilize that pre-existing interaction rather than drive a member interaction that has not yet occurred. Additionally, as will be understood, if the method is used to detect inhibitors or enhancers of the member binding interaction, then the oligonucleotide binding interaction should not be so great as to impact or interfere with such measurement.

In some embodiments, the concentration of the first member-oligonucleotide conjugate is about the same as the concentration of the second member-oligonucleotide conjugate. However, the disclosure also contemplates instances in which the first member-oligonucleotide conjugate may be used at a concentration that is different (i.e., less or more) than the concentration of the second member-oligonucleotide conjugate. The first member-oligonucleotide conjugate may have a concentration that is about 10%, or about 1000% of the concentration of the second member-oligonucleotide conjugate.

Another aspect of this disclosure provides a method for detecting an agent capable of modulating a binding interaction such as an inhibitor/antagonist or an enhancer/agonist. The method comprises providing (i) a first member of a binding pair, conjugated to a first oligonucleotide conjugated to a FRET donor fluorophore (and optionally referred to herein as a FRET donor oligonucleotide), and (ii) a second member of the binding pair, conjugated to a second oligonucleotide conjugated to a FRET acceptor fluorophore (and optionally referred to herein as a FRET acceptor oligonucleotide). The first and second members may bind to each other with a member dissociation constant, Kd, in the range of about 100 pM to about 100 PM. The first and second oligonucleotides may hybridize to each other with an oligonucleotide dissociation constant, Kd, that ranges from about 100 pM to about 1 μM and is comparable to or greater than the concentration of oligonucleotides (and accordingly the concentration of the member-oligonucleotide conjugates) used in the assay, including but not limited to about 100 pM to about 100 nM. The method further comprises combining the first member and the second member with a sample, under conditions that allow binding of the first member to the second member and that allow binding of the first oligonucleotide to the second oligonucleotide. The final concentrations of each member (or each member-oligonucleotide conjugate, or each oligonucleotide) may be in the range of about 100 pM to about 100 nM, provided it is below the oligonucleotide Kd, in this and other assays described in this disclosure. Test fluorescence from the FRET acceptor (i.e., fluorescence at the emission wavelength of the FRET acceptor) is then measured.

The sample may be identified as comprising an agent that decreases binding of the first member to the second member if the test fluorescence is less than a control fluorescence measured after combining the first member and the second member under the same conditions as but in the absence of the sample. Alternatively, the sample may be identified as comprising an agent that increases binding of the first member to the second member if the test fluorescence is greater than a control fluorescence measured after combining the first member and the second member under the same conditions as (b) but in the absence of the sample. If there is no significant difference between the test and the control fluorescence, then the sample may be determined to contain neither an inhibitor/antagonist nor an enhancer/agonist. It is expected that a sample or a plurality of samples will be tested, possibly concurrently in the context of a high throughput assay, for the presence of either an inhibitor/antagonist or an enhancer/agonist.

The control assay may be performed at the same time, and alongside the test assay. There may be a control assay for each test assay, or there may be one control assay for a plurality of test assays. Alternatively, the result of a control assay, such as a fluorescence measurement in the absence of a sample, may have been performed ahead of time and may be known for a certain set of components and conditions, and such value may be used as a comparator to a test fluorescence, without having to run a control assay every time a test assay is run.

Accordingly, the sample may be identified as containing an agent that modulates binding of the first member to the second member, wherein a test fluorescence that is less than a control fluorescence, measured in the absence of the sample, is indicative of an agent that decreases binding of the first member to the second member, and a test fluorescence that is greater than a control fluorescence, measured in the absence of the sample, is indicative of an agent that increases binding of the first member to the second member.

As noted above, the method may be carried out at one or more different member-oligonucleotide (or member, or oligonucleotide) concentrations, ranging for example from about 100 pM to about 100 nM. The oligonucleotides may be chosen or designed such that the oligonucleotide Kd is about 1 times, 2 times, 5 times, 10 times or higher than the concentration of oligonucleotides used in the assay. The first and second members may be provided at concentrations lower than the member Kd, optionally 10-fold lower, 50-fold lower, 100-fold lower, 500-fold lower, or 1,000-fold lower, or 5,000-fold lower.

The samples to be tested may be naturally occurring samples, such as human samples or companion animal (e.g., dog, cat, etc.) samples, or agricultural animal (e.g., cow, swine, etc.) samples. They may be a bodily fluid sample such as blood or urine, or they may be derivatives thereof such as serum. In some embodiments, the sample is a human serum sample, optionally taken from a human subject who has received one or more vaccinations against a particular pathogen such as a virus, or a human subject who has had an infection.

Other aspects of this disclosure provide a system and a method for detecting agents that inhibitor or interfere with binding of a virus and the cell to which it binds. The virus may be a human virus and the cell may be a human cell. The method may use the virus and cell as the components, or if the particular binding partners are known, it may involve the particular viral component that binds to the cell and the particular cellular component to which it binds. As an example, the viral component may be Sars-CoV-2 receptor binding domain (RBD) and the cellular component may human receptor ACE2. RBD is known to bind to ACE2 and this interaction is important to the infection of human cells by the Sars-CoV-2 virus. Agents that interfere with this binding, either completely or partially, can be detected and optionally their levels measured using the methods of this disclosure. Optionally, the assay can be performed using a dilution series of the sample, such as but not limited to diluting the sample 1:10, 1:100, 1:1000, 1:10000, 1:100000, and potentially higher. The presence of such agents, typically in the form of antibodies, in a human sample may indicate that the subject has some degree of immunity to the virus, whether by prior vaccination or prior infection. In addition to detecting presence of the antibody, the method may be used to determine the level of the antibody, and thereby provide information as to how well the subject is protected from re-infection. The method may be performed repeatedly at regular intervals to determine whether the subject should receive a booster dose. The RBD—ACE2 binding interaction has been reported to have a Kd of about 400 nM. The assay could be performed using, for example, oligonucleotides having a Kd of about 50 nM, and the member-oligonucleotide conjugate may be used in concentrations that are equal to or less than 50 nM, including for example at about 40 nM, about 30 nM, about 20 nM, about 10 nM, or lower.

The neutralization between ACE2 and wild type RBD is demonstrated in the Examples, but the approach could also be used to test inhibition of binding between ACE2 and mutant or variant forms of RBD or full-spike trimers, in a multiplexed manner.

The conditions under which the members (or member-oligonucleotide conjugates) are combined with each other, whether in the presence (test) or absence (control) of sample, may vary on the nature of the members as well as other factors. The assay may be performed at a range of temperatures including but not limited to from about 4° C. to about 37° C., or from about 4° C. to about 10° C., or from about 10° C. to about 18° C., or from about 18° C. to about 25° C., or from about 20° C. to about 22° C., or about room temperature. The assay may be performed at about 4° C., or at about 22° C., or at about 37° C. The assay may be performed for a period of time sufficient for both the members and the oligonucleotides to form their respective binding interactions. The time may range, for example, from 5 minutes to 2 hours, or from 10 minutes to 1 hour, or from 20 minutes to 1 hour, or from 30 minutes to 1 hour, or it may be about 30 minutes. In some embodiments, the conditions comprise a temperature of about room temperature (e.g., in the range of about 20° C. to about 22° C.) and the time is about 30 minutes.

The methods provided herein may be performed as part of a high throughput assay, conducted using small volume reactions, and low concentrations of components. In this way, the assays may be conducted in short periods of time and for comparatively low cost, given the lower levels of components that are required. The platform may be a multiwell plate such as but not limited to a 384-well plate, with each assay being performed in a single well. An assay may be performed in duplicate, or triplicate, or more time per well and an average fluorescence measurement from such wells may be used as the test fluorescence. Similarly, the multiwell plate may also have one, two, three or more wells devoted to the control assay (e.g., without sample), and an average fluorescence measurement from such wells may be used as the control fluorescence.

Another advantage of the methods provided herein is the ability to perform them without the need to perform washing between consecutive steps, particularly between the step combining and incubating the components (with or without the sample) and the step of measuring fluorescence.

The components are also typically in free form (i.e., in solution) rather than bound to a relatively inflexible solid surface such as the well bottom or well side or a relatively inflexible solid support such as a bead. As discussed herein, the components may all be conjugated to a single flexible polymer, but this is still considered according to this disclosure to be in free form (i.e., in solution).

It will be understood as a result of this disclosure that the FRET oligonucleotides should be attached to their respective members at locations that render the FRET oligonucleotides accessible and available for hybridization to each other. The positioning of each FRET oligonucleotide on its respective member relative to the location of the member binding interaction will depend on each conjugate, and can be readily determined by the person of skill based on this disclosure. In embodiments relating to polymer-FRET, discussed in greater detail herein, wherein the first and second members are attached to a single polymer, the distance between the positions of the first and second members on the single polymer should be sufficient to allow binding of the first and second members to each other. The distance that is sufficient will depend in part on the flexibility of the polymer and the size of the members.

Another aspect of this disclosure provides a system and a method for analyzing modulation of a binding interaction using fluorescence polarization.

Fluorescence polarization (FP) is a method known in the art for assaying biochemical interactions. In theory, FP is related to how molecules rotate freely in three-dimensional space. For example, a small molecule rotates fast in solution, whereas a larger molecule rotates slower relative to the smaller molecule under the same conditions. Therefore, when a fluorescently labelled compound is excited by linearly polarized light, the polarization of the fluorescence that is emitted increases with the molecular weight of the labelled molecule, due to reduced rotation. This makes FP a suitable approach for characterizing biochemical interactions as the size of the complex attached to the fluorophore increases when interactions occur between two molecules such as, but not limited to, formation of a protein-protein interaction. Reference can be made to Hall et al. 2016, U.S. Pat. Nos. 6,432,632; 77,803,554 B2.

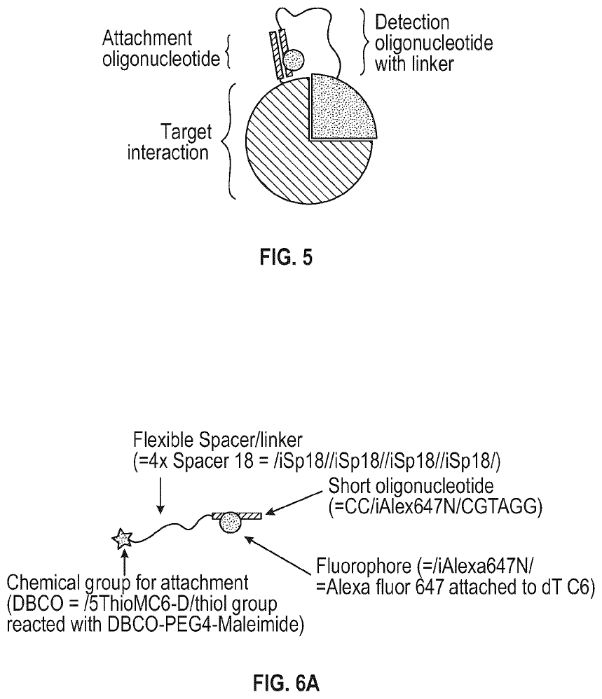

The FP system contemplated by this disclosure may comprise and the method use (1) a first member of a binding pair, conjugated to a first oligonucleotide, wherein the first oligonucleotide is conjugated to a fluorophore, and (2) a second member of the binding pair, conjugated to a second oligonucleotide. The fluorophore conjugated to the first oligonucleotide is not necessarily a FRET donor or acceptor fluorophore, as this system and method are not reliant on FRET to detect close proximity of the oligonucleotides, and thus the member binding partners. Similarly, in this embodiment, the second oligonucleotide is not necessarily conjugated to a fluorophore. The fluorescent signal may be that emitted from the fluorophore on the first member-oligonucleotide conjugate. The first and second members may bind to each other with a member dissociation constant, Kd, in the range of about 100 pM to about 100 μM. The first and second oligonucleotides may hybridize to each other with an oligonucleotide dissociation constant, Kd, that ranges from about 100 pM to about 1 μM and preferably is comparable to or greater than the concentration of oligonucleotides (or member-oligonucleotide conjugates) used in the assay including but not limited to about 100 pM to about 100 nM. In contrast to the other systems and methods provided herein, particularly those that are referred to as linker-FRET systems and methods, the first member is conjugated to the first oligonucleotide using a first linker but the second member may be conjugated to the oligonucleotide directly or with a much shorter linker than used for the first member-oligonucleotide conjugate. Accordingly, and as illustrated in FIG. 5, one oligonucleotide is relatively rigidly positioned on its respective member, while the other by virtue of the linker (or longer linker) has greater movement. It is the hybridization of the two oligonucleotides that then renders the fluorophore relatively rigid in its position. The difference in fluorescent signal from the unbound and the bound fluorophore can be detected using FP. As with other embodiments described herein, the first linker may have a length of about 2-14 nm, although it is not so limited, and provided that the second linker, if even present, is sufficiently shorter to impart rigidity to the second oligonucleotide as compared to the first oligonucleotide. The first linker may be but is not limited to 1 to 6 adjacently (or consecutively) order hexa-ethyleneglycol units such as 18-atom hexa-ethyleneglycol units.

Another aspect of this disclosure provides an FP-based method for detecting an agent capable of modulating a binding interaction. The method comprises (a) providing (i) a first member of a binding pair, conjugated to a first oligonucleotide, wherein the first oligonucleotide is conjugated to a fluorophore, and (ii) a second member of the binding pair, conjugated to a second oligonucleotide, wherein the first and second members bind to each other with a member dissociation constant, Kd, in the range of about 100 pM to about 100 μM, and wherein the first and second oligonucleotides hybridize to each other with an oligonucleotide dissociation constant, Kd, that ranges from 100 pM to 1 μM and is comparable to or greater than the concentration of oligonucleotides (or member-oligonucleotide conjugates) used in the assay, which may optionally be about 100 pM to about 100 nM, (b) combining the first member and the second member with a sample, under conditions that allow binding of the first member to the second member and that allow binding of the first oligonucleotide to the second oligonucleotide, (c) measuring test fluorescence using FP, and (d) identifying the sample as containing an agent that (i) decreases binding of the first member to the second member as a test fluorescence that is less than a control fluorescence measured after combining the first member and the second member under the same conditions as (b) but in the absence of the sample, or (ii) increases binding of the first member to the second member as a test fluorescence that is greater than a control fluorescence measured after combining the first member and the second member under the same conditions as (b) but in the absence of the sample. This method could be performed in wells of a multiwell plate, and would be amenable to high-throughput. The input energy would be polarized light at an excitation wavelength specific to the fluorophore, and the output would be the degree of polarization of the emitted light, which may be expressed in units of 1 P (or 1000 mP) indicating full polarization, and is often in the range of 10-300 mP.

Still other aspects of this disclosure are directed to polymer-FRET systems and methods. In polymer-FRET, the members are attached to a single polymer (or a single complex), and the polymer (or complex) is sufficiently flexible to allow the members to interact and bind to each other. The members may or may not be conjugated to oligonucleotides, such as FRET oligonucleotides, or FRET oligonucleotides may be bound to the polymer in close proximity to the members. For example, in some instances, member-oligonucleotide conjugates may be bound to the polymer, and when the members come together and create a binding interaction, the oligonucleotides will also hybridize and thereby stabilize the member-member binding interaction. If the oligonucleotides are FRET oligonucleotides, as described herein, then their hybridization results in a FRET signal (i.e., fluorescence emission from the FRET acceptor). The positioning of the members (and the oligonucleotides) on a single polymer increases their relative “spatial” (or effective local) concentration without the need for more members or oligonucleotides or fluorophores, any and all of which may be costly.

One aspect of the polymer-FRET applications is directed to a system for analyzing modulation of a binding interaction. The system comprises a polymer conjugated to a first member of a binding pair and a second member of a binding pair, wherein the first and second members bind to each other with a member dissociation constant, Kd, of about 100 pM to about 100 μM. The polymer is sufficiently flexible to adopt a different secondary conformation that facilitates the members coming in proximity with each other. This may occur by having the polymer adopt a looped conformation when the first and second members bind to each other. The polymer is further conjugated to a first FRET oligonucleotide conjugated to a first FRET fluorophore and a second FRET oligonucleotide conjugated to a second FRET fluorophore, wherein the first and second FRET oligonucleotides hybridize to each other with a FRET oligonucleotide dissociation constant, Kd, which in some embodiments may be comparable to (about equal to) or greater than the concentration of the polymer (and thus the members and thus the oligonucleotides) used in the assay, and which in some embodiments may be comparable to or greater than the effective local concentration between the oligonucleotides on the polymer. As will be understood, one of the FRET fluorophores is a FRET donor fluorophore and the other is a FRET acceptor fluorophore.

The polymer may be a nucleic acid, or a linear amino acid chain, or polyethylene glycol. It should be sufficiently long and not capable of adopting secondary structure, such as hairpins in the case of nucleic acids or a globular form in the case of amino acids. Preferably, it is a linear polymer of sufficient length.

A related aspect of this disclosure provides a method for detecting an agent capable of modulating a binding interaction using the polymer-FRET methodology described above. The method comprises (a) combining the foregoing polymer construct with a sample, under conditions that allow binding of the first member to the second member and that allow binding of the first FRET oligonucleotide to the second FRET oligonucleotide, (b) measuring test fluorescence from the FRET acceptor, and (c) identifying the sample as containing an agent that (i) decreases binding of the first member to the second member as a test fluorescence that is less than a control fluorescence measured after incubating the polymer under the same conditions as (a) in the absence of the sample, or (ii) increases binding of the first member to the second member as a test fluorescence that is greater than a control fluorescence measured after incubating the polymer under the same conditions as (a) in the absence of the sample.

Another aspect of the polymer-FRET applications is directed to a system for analyzing modulation of a binding interaction, whereby the system makes use of nucleic acid complexes referred to herein as nanoswitches. These nucleic acid complexes or nanoswitches comprise a comprising a single-stranded scaffold nucleic acid hybridized to a plurality of single-stranded oligonucleotides thereby forming a linear partially double-stranded nucleic acid. These complexes have been described in U.S. Pat. No. 9,914,958, the contents of which relating to nanoswitches, their composition and their methods of making are incorporated by reference herein. The nanoswitches of the present disclosure may comprise a scaffold that is full length M13 (about 7200 bases, and about 2400 nm), or that is a fragment thereof, and therefore may have a length of about 20 nm, or about 50 nm, or about 100 nm, or about 500 nm, or about 1000 nm, or about 2500 nm, for example. Accordingly, the scaffold need not be as long as the scaffold in a nanoswitch that requires a gel electrophoresis read out, as described in the '958 patent. Rather, it may be about 100 nm to about 2500 nm, or about 100 nm to about 1000 nm, or about 100 nm to about 500 nm, or about 500 nm to about 1000 nm, provided the members and oligonucleotides positioned thereon are able to interact with these other.

In these systems, a first single-stranded oligonucleotide in the plurality is linked to a first member of a binding pair and a second single-stranded oligonucleotide in the plurality is linked to a second member of a binding pair, wherein the first and second members bind to each other with a member dissociation constant, Kd, of about 100 pM to about 100 μM. Further, a first FRET oligonucleotide, comprising a first FRET fluorophore, is conjugated to the complex (or nanoswitch) and a second FRET oligonucleotide, comprising a second FRET fluorophore, is conjugated to the complex (or nanoswitch), wherein the first and second FRET oligonucleotides hybridize to each other with a FRET oligonucleotide dissociation constant, Kd, that is comparable to or greater than the concentration of the FRET oligonucleotides (or members, or nanoswitches) in the assay. One of the FRET fluorophores is a FRET donor fluorophore and the other is a FRET acceptor fluorophore.

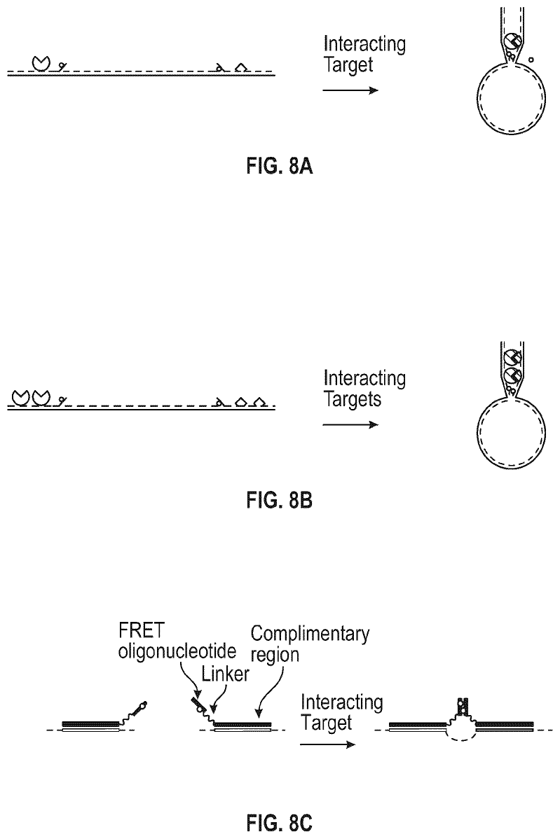

In some embodiments of the nanoswitch-FRET system and method, the first FRET oligonucleotide is conjugated to the complex (nanoswitch) using a first linker and/or the second FRET oligonucleotide is conjugated to the complex using a second linker, optionally wherein the first and/or second linker comprises one or more hexa-ethyleneglycol spacers, such as 18-atom hexa-ethyleneglycol spacers. In other words, the first FRET oligonucleotide and/or the second FRET oligonucleotide may be conjugated to the polymer or the nanoswitch, but not to the first or second member. This is illustrated in the FIG. 8A. Therefore, in polymer-FRET or nanoswitch-FRET the members and oligonucleotides may be conjugated to each other, or they may be conjugated to the polymer or nanoswitch. In either configuration, both have a higher effective local concentration because of their attachment to the polymer or nanoswitch.

It is to be understood that these systems and methods comprise oligonucleotides that are bound to the scaffold (which may be referred to as tile oligonucleotides for clarity), and FRET oligonucleotides. The FRET oligonucleotide may be conjugated to a tile oligonucleotide directly or indirectly through the use of a linker such as a hexa-ethyleneglycol linker. The oligonucleotides are positioned along the length of the complex (i.e., along the length of the scaffold nucleic acid) in order, optionally as first oligonucleotide, first FRET oligonucleotide, second FRET oligonucleotide, and second oligonucleotide, thereby providing an order of first member, first FRET oligonucleotide, second FRET oligonucleotide, and second member, along the length of the scaffold. The distance between the first oligonucleotide and the first FRET oligonucleotide may be about 30 base pairs, in some instances. The distance between the second FRET oligonucleotide and second oligonucleotide may be about 30 base pairs, in some instances. The distance between the first FRET oligonucleotide and the second FRET oligonucleotide may be about 30 base pairs, about 100 base pairs, about 500 base pairs, about 1000 base pairs, or more provided the length allows the unimpeded contact of the members to each other and the FRET oligonucleotides to each other.

The complex is designed such that, some embodiments, the first and second FRET oligonucleotides bind to each other (or remain discernably bound to each other) only upon binding of the first and second members to each other.

In still other embodiments, the complex may comprise additional tile oligonucleotides that flank the first and second oligonucleotides. Such flanking oligonucleotides (e.g., a third and a fourth oligonucleotide) may be conjugated to the first member and the second member, respectively. This embodiment is illustrated in FIG. 8B. These embodiments allow monitoring and measuring of binding avidity between a first and a second member.

A related aspect of this disclosure provides a method for detecting an agent capable of modulating a binding interaction using the polymer-FRET methodology described above. The method comprises (a) combining any of the foregoing complexes with a sample, under conditions that allow binding of the first member to the second member and that allow binding of the first FRET oligonucleotide to the second FRET oligonucleotide, (b) measuring test fluorescence from the FRET acceptor, and (c) identifying the sample as containing an agent that (i) decreases binding of the first member to the second member as a test fluorescence that is less than a control fluorescence measured after incubating the complex under the same conditions as (a) in the absence of the sample, or (ii) increases binding of the first member to the second member as a test fluorescence that is greater than a control fluorescence measured after incubating the complex under the same conditions as (a) in the absence of the sample.

This disclosure contemplates that any of the assays provided herein may be performed in a multiplexed manner using different FRET pairs coupled to different binding partner pairs.

Compared to the direct labeling of proteins with fluorophores, the coupling of fluorescently-labeled DNA linkers to proteins has a number of advantages. First, it enables the optimization of a FRET signal: positioning the FRET partners on DNA oligos that complement gives precise positional control over the positions of these dyes, enabling the strength of the fluorescent signal to be maximized. Second, the binding energy between complementary DNA linkers can be engineered to tune the effective strength of protein-protein interactions of interest. By introducing an additional binding partner that binds in parallel to the proteins, interactions can be enhanced in a controllable way through an avidity effect, enabling the study of weak interactions. As the interaction strength is tunable, it enables the signal-to-noise ratio to be maximized, e.g. increasing the binding energy between complementary DNA linkers increases the fraction of bound proteins, increasing the signal, decreasing the binding energy between complementary DNA linkers decreases background FRET.

Even compared to other systems that use complementary DNA linkers, the unique optimization procedures described herein lead to a distinct advantage. Linker-FRET includes the use of short linkers (shorter than used previously), directly attached to the binding partners, potentially at specific locations that are in close proximity when the binding partners are bound. This enables the assaying of interactions using concentrations of binding partners that are much lower than their dissociation constants. Thus less material is needed, and this is key for lowering costs of high-throughput screening, or a neutralization assay such as for COVID

### EXAMPLES

### Example 1. Linker-FRET for In Vitro Screening of Protein-Protein Interaction Modulators

Introduction

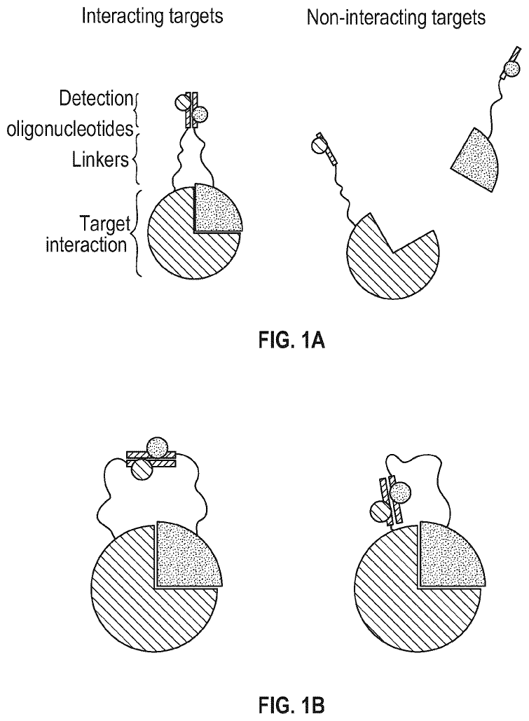

In linker-FRET two target interacting partners (which may be referred to herein as binding partners, targets, or members, interchangeably) are labeled (or conjugated, as the terms are used interchangeably herein) with oligonucleotides bearing a FRET donor or a FRET acceptor fluorophore (referred to herein as FRET oligonucleotides). One or both of these binding partner-oligonucleotide (or member-oligonucleotide) conjugates may have a linker (also referred to has a tether) between the binding partner and the oligonucleotide. As shown in FIG. 1A, the binding partners (denoted as targets, examples of which are proteins) are attached to FRET (or detection) oligonucleotides through a linker. When the binding partners interact, the FRET (or detection) oligonucleotides bind to each other and result in emission of a FRET signal, while if the binding partners do not interact, the FRET (or detection) oligonucleotides do not bind (hybridize) to each other and no FRET signal is detected.

The affinities of the FRET oligonucleotides and the lengths of the linkers are such that the fluorophore-containing oligonucleotides only substantially interact when the binding partners interact. The affinities of the FRET oligonucleotides and the lengths of the linkers are also designed to enable the interaction of binding partners that would otherwise not interact, at the concentration chosen for the assay, without the enhancement of binding caused by the FRET (or detection) oligonucleotides. The linker length should be a sufficient length to facilitate and promote hybridization of the FRET oligonucleotides, including short enough to significantly enhance the interactions of the binding partners. The linkers should be attached to the binding partners at positions that are in close proximity when the binding partners interact. The binding affinity of the FRET oligonucleotide, in some embodiments, is just below that which causes oligonucleotide hybridization in the absence of any binding partner interaction. Various orientations of linker-FRET are shown, but are not limited to, those in FIG. 1B. The Examples demonstrate that suitable Z factors, indicative of assay quality, are achievable using linker-FRET in a high throughput setting.

As an example of the robustness of linker-FRET, the Examples further demonstrate that this methodology can be used as the basis of a low cost assay for detecting and optionally measuring neutralizing antibodies against Sars-CoV-2 in human patient samples. In the presence of neutralizing antibodies present in a sample obtained from a patient, the interaction between the human receptor ACE2 and the Sars-CoV-2 receptor-binding domain (RBD) will decrease. This assay was performed in solution (free-form, unbound to a support or surface) and required lower concentrations of both binding partners than the dissociation constant between ACE2 and RBD, which is estimated to be ˜400 nM (Ramanathan et al. 2021). Therefore, the assay provided herein will be more cost-effective than currently available neutralization assays that are ELISA-based (Tan et al. 2020), or bead-based (Mravinacova et al. 2021, Fenwick et al. 2021), or comparable commercial assays (Krüttgen et al. 2021).

A linker system similar to that for linker-FRET can also be used to enhance FP, and is referred to herein as linker-FP. In this case, as illustrated in FIG. 5, only a single oligonucleotide probe is labeled with a fluorophore. Similar to linker-FRET, the oligonucleotides enable weakly interacting binding partners to interact at low concentrations. Different configurations of the fluorophore and coupled oligonucleotides can be used to maximize the change in the free motion of the fluorophore upon binding. These include, but are not limited to, a short oligonucleotide on the smaller protein with the fluorophore close to the protein attachment point and short linkers or rigid attachments between the oligonucleotide and protein, and oligonucleotide and fluorophore, along with the other binding oligonucleotide attached to the larger protein with a long flexible linker; or an oligonucleotide with a rigidly attached fluorophore on the end attached to the smaller protein through a long flexible linker, along with the other binding oligonucleotide attached to the larger protein with through a short, potentially rigid, attachment. As is shown in FIG. 5, upon binding of the smaller protein to the larger protein, the fluorophore at the end of a flexible linker and oligonucleotide is stabilized in position relative to the larger protein upon hybridization since the fluorophore has a rigid or short (potentially smaller than 4 nm) attachment to the coupled oligonucleotide and the binding oligonucleotide has a potentially short and/or potentially rigid attachment to the larger protein. Fluorophores with long life times can be used to maximize the FP signal difference upon binding.

In various of the exemplifications provided herein, short DNA oligonucleotides were used as model weakly interacting binding partners. However, it is to be understood that other binding partners may be assayed including, but not limited to small molecules, proteins, and peptides.

### Methods

### Linker-FRET Constrict Coupling

Oligonucleotides with Amino Modifier C6 dT were ordered from Integrated DNA technologies (Table 1). The oligonucleotides were coupled to amine reactive Alexa 555 or Alexa 647 fluorophores (ThermoFisher) overnight and then PAGE purified. The concentration was then quantified by absorption on a UV-vis machine. P_F and P_R were ordered already coupled to fluorophores and HPLC purified.

TABLE 1
Oligonucleotide sequences for linker-FRET

| Name | Sequence |
|---|---|
| Z3_F | CC\iAmMC6T\ACGAGG\iSp18\\iSp18\\iSp18\\iSp18\GTGTCC |
| Z3_R | GGACAC\iSp18\\iSp18\\iSp18\\iSp18\CC\iAmMC6T\CGTAGG |
| Z4_F | CC\iAmMC6T\ACGAGG\iSp18\\iSp18\\iSp18\\iSp18\ATGTCC |
| Z4_R | GGACAT\iSp18\\iSp18\\iSp18\\iSp18\CC\iAmMC6T\CGTAGG |
| Z5_F | CC\iAmMC6T\ACGAGG\iSp18\\iSp18\\iSp18\\iSp18\TGTCC |
| Z5_R | GGACA\iSp18\\iSp18\\iSp18\\iSp18\CC\iAmMC6T\CGTAGG |
| P_F | CC/iAlex555N/ACGAGG/iSp18//iSp18//iSp18//iSp18//3ThioMC3-D/[Mal-peg4-DBCO] |
| P_R | [Mal-peg4-DBCO]/5ThioMC6-D//iSp18//iSp18//iSp18//iSp18/CC/iAlex647N/CGTAGG |
| BL_F5 | CCTCGTAGG |
| BL_Z3 | GGACAC |
| BL_Z4 | GGACAT |
| BL_Z5 | rGrGrArCrA |

TABLE 2
Additional details on the composition of the molecular constructs

| Symbol | Description | Structure |
|---|---|---|
| iSp18 | Int Spacer 18 Flexible 18-atom hexa-ethyleneglycol spacer (is also known as PEG 6 = 6 polyethyleneglycol units) | 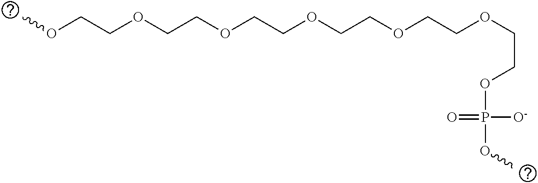 |
| iAmMC6T | Int Amino Modifier C6 dT Thymine (T) base that has a free amine group that can be attached to a fluorophore that is purchased with a NHS Ester modification | 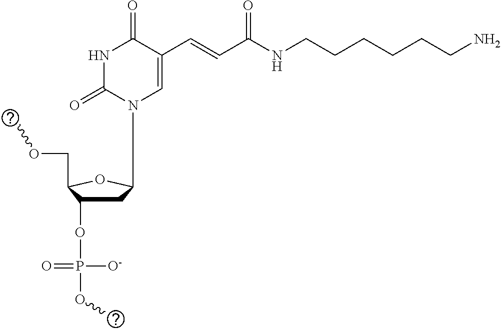 |
| iAlex555N; | The above C6 dT | |
| iAlex647N | attached to an | |
| | ALEXA FLUOR ® | |
| | 647 or ALEXA | |
| | FLUOR ® 555. | |
| 3ThioMC3-D | 3′ Thiol Modifier C3 S-S 3′ thiol modification | 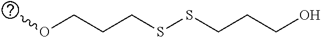 |
| | that reduces to SH in | |
| | order to react with | |
| | Maleimide chemistry. | |
| 5ThioMC6-D | 5′ Thiol Modifier C6 S-S 5′ thiol modification that reduces to SH in order to react with Maleimide chemistry. | 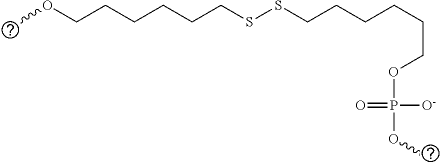 |

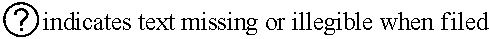

### Quantification of FRET and Z′

The purified constructs were diluted in assay buffer (20 mM Tris, pH 8.0, 10 mM MgCl2, 100 mM NaCl, 0.05% Tween-20) to twice the final assay concentration. Then they were mixed 1:1 with samples containing only buffer, or blocking oligonucleotide at 250 μM. The samples were then incubated for 30 minutes at room temperature and then 7 μl was aliquoted out into 384-well plates. FRET was quantified using a BioTek Neo plate reader. Specifically, the FRET emission was measured at 680 nm from excitation at 540 nm and the non-FRET emission at 555 nm from excitation at 540 nm using the built in monochromator-based system. The FRET ratio was then calculated as the ratio of the signal at 665 nm over 555 nm. Z′ is defined as 1-3(σs+σb)/(μs−μb), where σs is the standard deviation of the sample σb is the standard deviation of the background, μs is the average signal of the sample, and μb is the average signal of the background (Zhang et al. 1999). These values were calculated over 5 wells on a single plate for each condition. The background is in the presence of an oligonucleotide blocking the target interaction and the signal is the absence of any additional oligonucleotide.

### Coupling of Linker-FRET Probes to Proteins and Neutralization Assay

RBD and ACE2 were purchased from Sinobiological and coupled to the probes, P_F and P_R, respectively, using azido-peg4-NHS ester (Sigma). Uncoupled probes were removed using his-tag purification of the proteins. COVID+ serum from patients that have recovered from COVID-19 was purchased from Lee Biosolutions. To determine the effect of antibody spike-in or serum on the FRET signal, 20 nM of each coupled protein was mixed in and incubated for 30 minutes.

### Additional Details on the Procedure for Constructing and Using Molecular Constructs

1) Prepare the linker-FRET probes. Linker-FRET probes need to be constructed that are composed of a short oligonucleotide with an internal fluorophore attached to a flexible spacer/linker that has a chemical group for attachment at the end. In this version IDT manufactured the short oligonucleotide directly attached to the flexile spacer/linker of 4×6 unit PEG modifications and then also directly attached to a thiol group. The short oligonucleotide also has a dT C6 for attachment of the fluorophore. IDT then reacted the fluorophore with the free amine on the internal dT C6 nucleotide and purified using High-performance liquid chromatography (HPLC) to purify the fully coupled and manufactured constructs (gel purification can also be used). Next, the thiol group is attached to a dibenzocyclooctyne group (DBCO) by reacting the oligonucleotide with the reducing agent TCEP (tris(2-carboxyethyl)phosphine) for 2 hours so all the sulfhydryl group are available for coupling (reducing breaks the S—S bonds to 2×S—H), and then mixing with DBCO-PEG4-Maleimide for 2 hours, in which the Maleimide groups covalently coupled to the sulfhydryl groups, leaving a DBCO group available on the end of the probe. This DBCO group is the chemical group for attachment to the protein. The probe is then purified, such as by HPLC. The flexible spacer/linker can also composed partly or only of a longer peg molecule, such as DBCO-PEG24-Maleimide or DBCO-PEG24-NHS ester, that reacts with a terminal group, such as thiol or amine, respectively, on the oligonucleotide probe.

2) Couple the linker-FRET probes to the protein. Each protein is prepared to attach to its corresponding linker-FRET probe by modifying the protein with a chemical group that will react with the chemical group for attachment on the linker-FRET probe. Here the amine-reactive “Azido-PEG4-NHS ester” was used to attach azido groups nonspecifically to surface lysines or the N-terminus by mixing and incubating for 2 hours. Other options include coupling to natural or engineered—in cysteines using maleimide chemistry. Unreacted “Azido-PEG4-NHS ester” is then removed using a Zeba Spin Desalting Column. The resulting prepared protein is then mixed with the linker-FRET probe and incubated for 2 hours. The copper-free click reaction attaches the azido group on the protein with the dibenzocyclooctyne group (DBCO) group on the linker-FRET probe. Coupled proteins are purified away from uncoupled fret-linker probes. This can be performed by purifying the protein using a purification tag that was genetically engineered into the proteins, such as a his-tag in this case.

3) Perform the FRET experiment. The FRET experiment is performed by mixing together the 2 types of proteins that are coupled to reverse complimentary linker-oligonucleotides, along with the sample to be assayed. The concentrations of protein-linkers are approximately 0.1-100 nM. The sample to be assayed can be a human sample, such as serum that may contain antibodies, or a putative drug compound. After an incubation (usually 15 minutes to 1 hour), the samples are then read out on a plate reader to get the FRET signal, which is compared to a control sample with no compounds that affect the interaction between the 2 proteins and also potentially control samples with compounds known to decrease or increase the interaction between the 2 proteins.

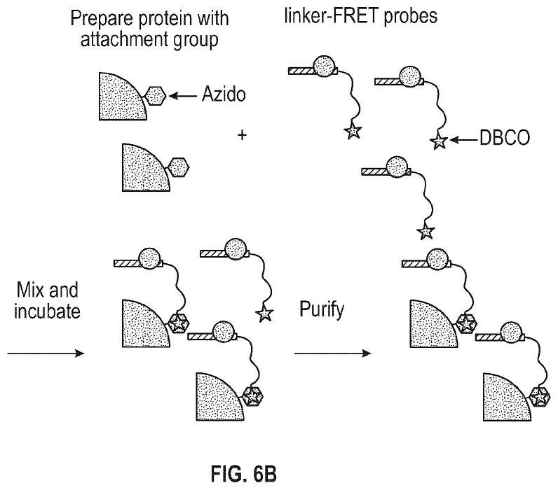

FIGS. 6A-6B provide schematics detailing the preparation procedure for linker-FRET with proteins as binding pairs. FIG. 6A shows a schematic of the linker-FRET probe. The 4 main components are the (1) short oligonucleotide (e.g., a 7-12 mer) with (2) fluorophore internally attached, (3) flexible linker/spacer, and (4) chemistry group for attachment to the protein. FIG. 6B shows a schematic of attachment of the protein to linker-FRET probe with steps including (1) preparing the protein by attaching a chemical group that will attach to the linker-FRET probe, (2) mixing with the prepared protein with the linker-FRET probe and incubating, and (3) purifying the protein away from free uncoupled linker-FRET probes.

### Results

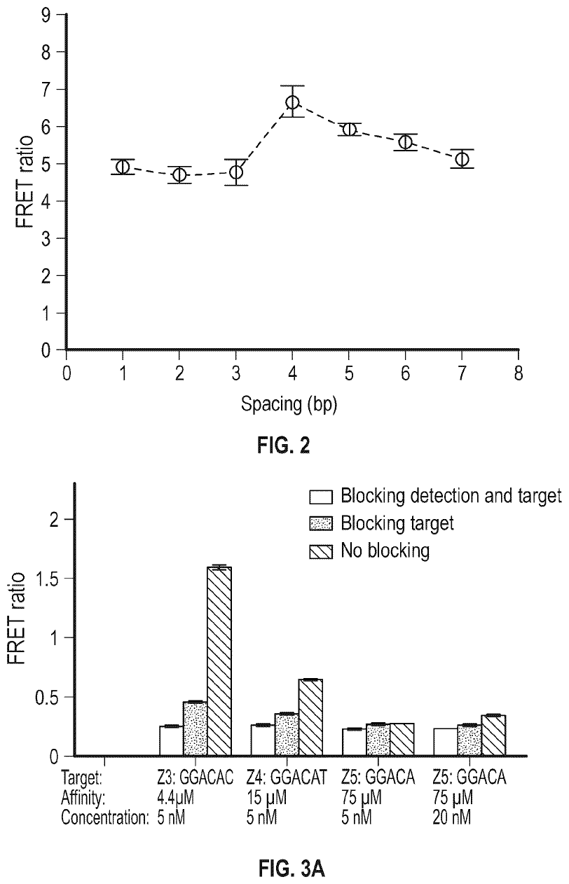

The effect of fluorophore spacing was tested on oligonucleotides on the FRET ratio of emission at 665 nm to 590 from an excitation at 555 nm. To do this 27mer oligonucleotides were used with different spacing between the fluorophores on the opposite strands. It was found that a spacing of 4-5 base pairs was optimal. FIG. 2 shows FRET ratio of emission at 665 nm (Alexa 647) to 590 nm (Alexa 555) as a function of fluorophore spacing. Alexa 555 and Alexa 647 were present on the amino modified C6 atom of dT bases. Oligo concentrations were 20 nM. Error bars are SEM over 4 replicates.

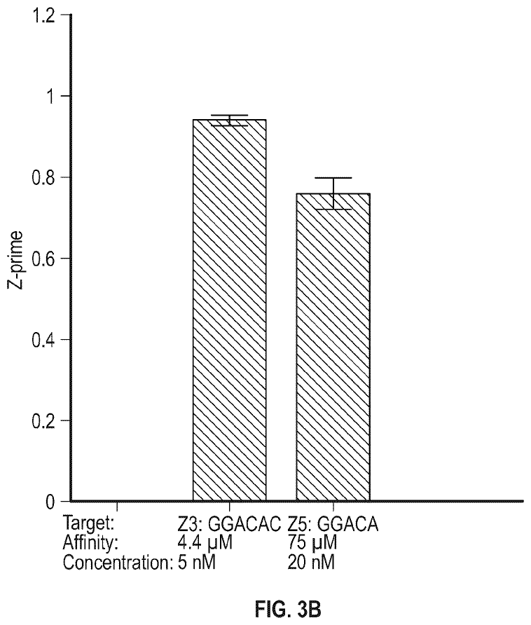

Next, linker-FRET was tested with the binding orientation shown in FIG. 3A, which short DNA oligonucleotides as the binding partners. The linkers here are composed of four adjacent 18-atom hexa-ethyleneglycol spacers, corresponding to a length of approximately 25 nm. When 5 nM of final concentration of probes were used, target interactions with predicted Kd's of 4.4 and 15 yielded a strong signal differential between blocking the target sequence and no blocking. FIG. 3A shows the background and signal from detection linker-FRET oligonucleotides. The ratio of emission at 665 nm to 590 nm of 5 nM or 20 nM of linker-FRET oligonucleotides with a V4 detection region, a linker composed of 4 18-atom hexa-ethyleneglycol spacers, and a target region specified from S3 to S5, which have decreasing predicted affinity from 4.4 μM to 75 μM. Results are given in the presence of short oligonucleotides that block both the detection and target oligonucleotides (white bars), short oligonucleotides the only block the target interaction (light grey bars), and the absence of any blocking oligonucleotides (dark grey bars). The blocking oligonucleotides were DNA and added to 50 μM, except for the blocking oligonucleotide for S5, which was RNA added at 250 μM. Oligo sequences are given in Table 1. Error bars are SEM over 2 replicates. Additionally, the signal for blocking only the target sequence is close to when you also block the FRET oligonucleotides sequence from interacting, indicating a low level of background signal due to interactions in the absence of any target-target binding. The signal for a target interaction with a predicted Kd of 75 does not have a strong signal differential though, but does when the concentration was increased to 20 nM.

The Z′ obtained when blocking the target oligonucleotide sequence was next measured. FIG. 3B shows assay quality Z-prime is obtained using linker-FRET for concentrations ranging from 5 nM to 20 nM for weak DNA-DNA target interactions of 4.4 μM or 75 μM. The background control is in the presence of 250 μM blocking oligonucleotide that binds to one part of the zipper, while the signal control is in the absence of any blocking oligonucleotide.

Z′ was calculated as 1−3(σs+σb)/(μs−μb) for each plate over 5 different points for each condition and the error bars are SEM Z′ is defined as 1−3(σs+σb)/(μs−μb), where σₛ is the standard deviation of the sample σ_{b} is the standard deviation of the background, μₛ is the average signal of the sample, and μ_{b} is the average signal of the background (Zhang et al. 1999). Z′ is a measure that combines the signal level above background and variability to quantify the statistical performance of an assay and assays with a Z′ of at least 0.5 are well-suited for HTS. It was found that using 5 nM of probe, Z′ reaches 0.94 for a target interaction with a Kd of 4.4 and using 20 nM of probe, Z′ reach 0.76 for a target interaction with a Kd of 75. This demonstrates that linker-FRET can be used to screen for enhancers or inhibitors of extremely weak interactions.

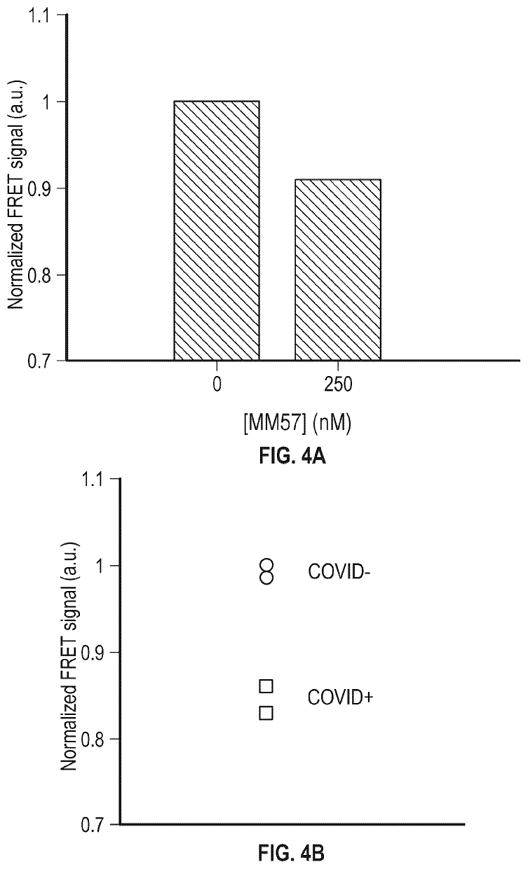

The ACE2:RBD system was next tested using 20 nM of each protein and saw that 250 nM of neutralizing antibody MM43 from Sinobiological decreased the FRET signal. FIG. 4A shows the normalized FRET signal for 0 nM compared to 250 nM of the known neutralizing antibody MM43 from Sinobiological 1:50 dilutions of 2 COVID-serum and 2 COVID+ serum Samples from patients that have recovered from COVID19 were also tested. FIG. 4B shows linker-FRET assay for 1:50 dilutions of 2 COVID-serum samples (circles) and 2 COVID+ serum samples from patients that have recovered from COVID19 (squares). Experiments were performed using 20 nM each of ACE2 coupled to P_R and RBD coupled to P_F. From these results a decrease in FRET for the COVID+ samples as compared to the COVID-samples was observed.

### DISCUSSION

Presented herein is linker-FRET for assaying modulation of weak molecular interactions in a format suitable for HTS. Using short complimentary DNA sequences, Z′ scores of approximately 0.75 were shown for interactions as weak as 75 μM.

### Example 2. Nanoswitch-FRET for In Vitro Screening of Molecular Interactions Modulators Such as Protein-Protein Interaction Modulators

Introduction

This disclosure contemplates using structural DNA nanotechnology to create programmable biomolecular materials capable of sensing, responding, and reporting changes in their local environments. Using such technology, nanoswitch-FRET has been developed to assay and discover compounds that modulate the interaction between two target proteins in a homogeneous HTS, with an emphasis on discovering compounds that increase protein-protein interactions (PPIs).

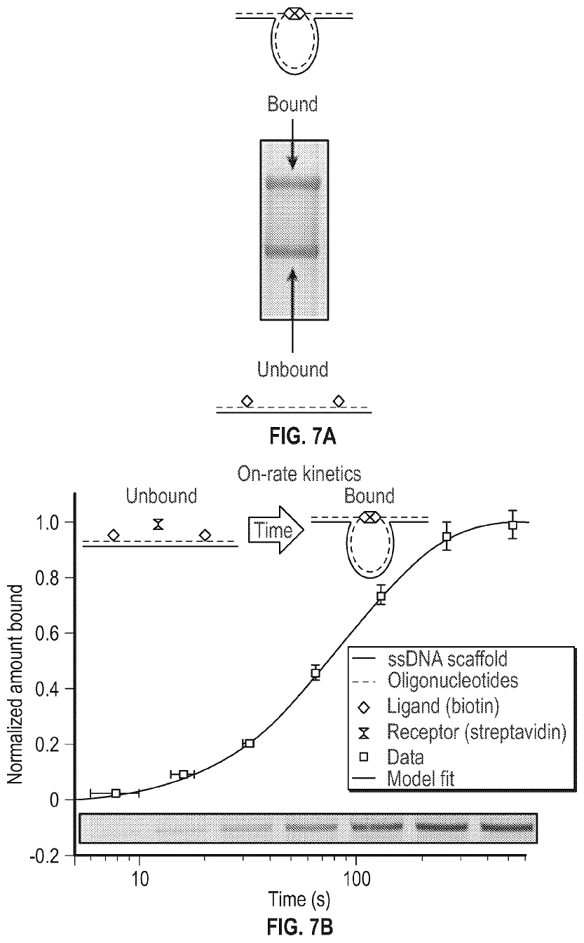

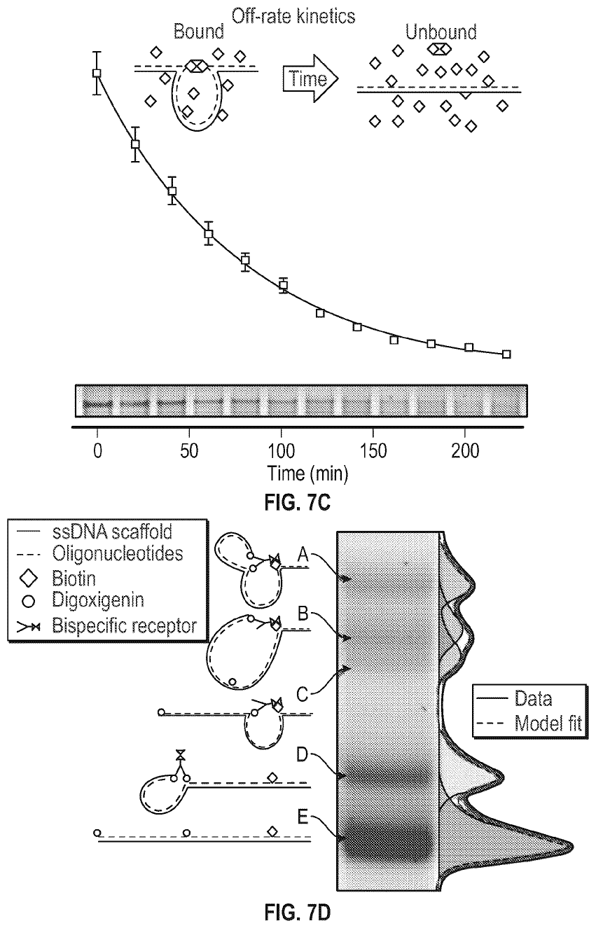

In nanoswitch-FRET, the use of FRET is demonstrated in combination with DNA nanoswitches for HTS of molecular interactions including weak molecular interactions. DNA nanoswitches were developed (Halvorsen et al. 2011) as a platform for biomolecular interaction analysis (Koussa et al. 2015) and analyte detection (Hansen et al. 2017) using a gel-based readout. As is shown in FIG. 7A, the two states (bound and unbound) of the DNA nanoswitches can be distinguished by gel electrophoresis. Nanoswitches may be typically made using linear M13 ssDNA with 2 or more target components (i.e., binding partners or members), such as proteins, peptides or oligonucleotides, conjugated at specific locations. When these target components interact with each other the linear DNA (scaffold) forms a loop. The presence of a loop can be read-out with gel electrophoresis since looped nanoswitches run more slowly than linear nanoswitches to create a distinct visual band. Nanoswitches can be used to character on and off-rates between molecules.

As is demonstrated in FIG. 7B, with two biotins integrated into the nanoswitch, loop formation begins when unlabeled streptavidin is introduced and progresses over time as evidenced by increasing intensity in the bound (looped) band across different lanes of a gel (bottom). The growth curve is fit with a kinetic model to determine the on-rate. As is shown in FIG. 7C, addition of excess biotin blocks loop formation, making bond rupture irreversible, which leads to the exponential decay of nanoswitches from the bound state to the unbound state, as shown by the decreasing intensity in the unbound band across different lanes of a gel (bottom).

Notably, nanoswitches can measure interactions of a multibody system. As is shown in FIG. 7D, a nanoswitch functionalized with two digoxigenin molecules and one biotin molecule can adopt five discernible states upon addition of a bispecific receptor. All five topological states, A-E, can be resolved within a single lane of an agarose gel. These bands can be fit globally with a single fit of a sum of skewed Gaussian curves. The black curve represents the median pixel intensity, the dashed curve represents the fit that is the sum of five skewed Gaussians, and the individual skewed Gaussians are shaded by state.

Here, the use of FRET is demonstrated as a readout for HTS using plate-readers. In nanoswitch-FRET, the formation of a loop brings fluorophores in close proximity to each other, resulting in an optical FRET signal from a homogenous assay (FIG. 8A). Compared to traditional FRET measurements, the nanoswitch keeps interacting partners together at a locally high concentration, so the effective concentration is higher than they would be free in solution. Additionally, the fluorophores for FRET are included onto short DNA sequences that only hybridize to each other when they are brought in close proximity as the loop closes. The zipper serves two purposes: it positions the fluorophores relative to each other for maximum FRET efficiency and also enhances the affinity of weak target interactions. This enables more cost-effective assaying of lower affinity interactions. Additionally, nanoswitches allow for easy attachment of more than two proteins can be used to study platform to study multibody interactions. This can also be used to include an avidity effect, in which multiple targets are attached to enable assaying very weak interactions (FIG. 8B). Presented herein, using weak DNA-DNA zipper interactions, the use of nanoswitch-FRET is demonstrated for HTS and characterization using a plate reader. Using nanoswitch-FRET, the Ki of inhibitors are measured and also Z-factors suitable for HTS are obtained.

A nucleic acid-based nanoswitch was used as a flexible linker/scaffold for nanoswitch-FRET, but other flexible, and relatively linear polymers can be used to connect the two binding partners and the fluorescent pairs, such as PEG or a polypeptide. A polymer or a nanoswitch, or the scaffold of the nanoswitch may have a length of about 20 nm, about 50 nm, about 100 nm, about 500 nm, about 1000 nm, or about 2500 nm. The length may be in the range of about 20 nm to about 2500 nm, or about 20 nm to about 1000 nm, or about 20 nm to about 500 nm, or about 20 nm to about 100 nm, or about 100 nm to about 500 nm, or about 100 nm to about 1000 nm.

One fluorophore pair and one or two binding partner pairs were used in these exemplifications but more binding partner pairs and/or fluorophore pairs could be used. Additionally, FRET pairs could also be attached to the nanoswitch/scaffold without intermediate linkers or they could be directly attached to the binding partners through a fluorescent protein fusion or conjugated fluorophore as described herein.

Additionally, two different DNA constructs can be used as a scaffold to hold together multiple proteins and/or fluorophores to interact for a signal instead of having both targets on the same DNA nanoswitch.

### Methods

### Nanoswitch Formation

DNA nanoswitches were created as previously described (Koussa et al. 2015, Hansen et al. 2017). Briefly, circular ssDNA from the 7,249-nt bacteriophage M13 (New England BioLabs) was linearized by enzymatic cleavage of a single site using BtscI (New England BioLabs) and a site-specific oligonucleotide. This ssDNA scaffold was mixed with a molar excess of tiling 60-mer oligonucleotides (10:1), excluding the complementary regions for detection oligonucleotide and target interaction partners, and subjected to a temperature ramp from 90 to 20° C. at 1° C.−min-1. The detection oligonucleotides were added at 55° C. during the hybridization protocol. All oligonucleotides were ordered from Integrated DNA technologies, with the detection oligonucleotides subjected to HPLC purification (Table 3). After hybridization, nanoswitches were purified from excess antibodies and oligonucleotides by using the BluePippin with a 0.75% Dye-Free agarose gel cassette, the Si marker, and the 6-10 k high-pass v3 protocol with a 4,500-bp cutoff. Alternatively, PEG precipitation (Koussa et al. 2015) or Fast protein liquid chromatography (Shaw et al. 2015) can be used for purification. For the short oligonucleotide zippers (Z1-Z3), the predicted Kd were obtained from I.D.T.'s oligoanalyzer tool.

### Incubation of Nanoswitch and FRET Quantification

The purified nanoswitches were diluted in assay buffer (10 mM HEPES, pH 7.5, 10 mM MgCl2, 100 mM NaCl) to twice the final assay concentration. Then they were mixed 1:1 with samples containing only buffer, 12 nM SA, or blocking oligonucleotide at 100 μM (unless otherwise specified), depending up the condition. The samples were then incubated for 1 hour at room temperature and then 7 μl was aliquoted out into 384-well plates.

### Quantification of FRET

The FRET from nanoswitches was quantified using a BioTek Neo plate reader. Specifically, the FRET emission was measured at 665 nm from excitation at 555 nm and the non-FRET emission at 555 nm from excitation at 555 nm using the built in monochromator-based system. The FRET ratio was then calculated as the ratio of the signal at 665 nm over 555 nm. For nanoswitch concentrations 2 nM and above, to reduce background, excitation at 540 nm was used instead and the FRET emission was measured at 680 nm.

### Quantification of Z′

Z′ is defined as 1−3(σs+(b)/(μs−μb), where σs is the standard deviation of the sample σb is the standard deviation of the background, μs is the average signal of the sample, and μb is the average signal of the background (Zhang et al. 1999). These values were calculated over 5 wells on a single plate for each condition. For biotin nanoswitches the signal is in the presence of SA and the background is in the absence of SA. For nanoswitches with short oligonucleotides as the target interaction, the background is in the presence of an oligonucleotide blocking the target interaction and the signal is the absence of any additional oligonucleotide.

TABLE 3
Oligonucleotide sequences for nanoswitch-FRET

| | | SEQ |
|---|---|---|
| | | ID |
| Name | Sequence | NO: |
| F1_A | TCAGTTGGCAAATCAACAGTTGAAAGGAAT\iSp18\C\iAlex647N\CGTAG | 1 |
| F1_B | C\iAlex555N\ACGAG\iSp18\GTTTTAGCGAACCTCCCGACTTGCGGGAG | 2 |
| F2_A | TCAGTTGGCAAATCAACAGTTGAAAGGAAT\iSp18\CT\iAlex647N\CGTAG | 3 |
| F2_B | C\iAlex555N\ACGAAG\iSp18\GTTTTAGCGAACCTCCCGACTTGCGGGAG | 4 |
| F3_A | TCAGTTGGCAAATCAACAGTTGAAAGGAAT\iSp18\CC\iAlex647N\CGAAG | 5 |
| F3_B | C\iAlex555N\TCGAGG\iSp18\GTTTTAGCGAACCTCCCGACTTGCGGGAG | 6 |
| F4_A | TCAGTTGGCAAATCAACAGTTGAAAGGAAT\iSp18\CC\iAlex647N\CGTAGG | 7 |
| F4_B | CC\iAlex555N\ACGAGG\iSp18\GTTTTAGCGAACCTCCCGACTTGCGGGAG | 8 |
| F5_A | TCAGTTGGCAAATCAACAGTTGAAAGGAAT\iSp18\GC\iAlex647N\CGAAGC | 9 |
| F5_B | GC\Alex555N\TCGAGC\iSp18\GTTTTAGCGAACCTCCCGACTTGCGGGAG | 10 |
| F6_A | TCAGTTGGCAAATCAACAGTTGAAAGGAAT\iSp18\GC\iAlex647N\CCGAGCG | 11 |
| F6_B | CGC\iAlex555N\CGGAGC\iSp18\GTTTTAGCGAACCTCCCGACTTGCGGGAG | 12 |
| F7_A | TCAGTTGGCAAATCAACAGTTGAAAGGAAT\iSp18\GCC\iAlex647N\CCGAGCG | 13 |
| F7_B | CGC\Alex555N\CGGAGGC\iSp18\GTTTTAGCGAACCTCCCGACTTGCGGGAG | 14 |
| F8_A | TCAGTTGGCAAATCAACAGTTGAAAGGAAT\iSp18\GGCC\iAlex647N\CCGAGCGC | 15 |
| F8_B | GCGC\iAlex555N\CGGAGGCC\iSp18\GTTTTAGCGAACCTCCCGACTTGCGGGAG | 16 |
| F5_avi_A | AATCAACAGTTGAAAGGAAT\Sp18\GC\iAlex647N\CGAAGC | 17 |
| F5_avi_B | GC\Alex555N\TCGAGC\iSp18\GTTTTAGCGAACCTCCCG | 18 |
| Biotin_A | CTCAAATATCAAACCCTCAATCAATATCT\3Bio\ | 19 |
| Biotin_B | \5Biosg\TTTTGAAGCCTTAAATCAAGATTAGTTGCT | 20 |
| Z1_A | CTCAAATATCAAACCCTCAATCAATATCTTTTTTTATGTCCAT | 21 |
| Z1_B | ATGGACATTTTTTTTTTTGAAGCCTTAAATCAAGATTAGTTGCT | 22 |
| Z2_A | CTCAAATATCAAACCCTCAATCAATATCTTTTTTTTTTATGTCCA | 23 |
| Z2_B | TGGACATTTTTTTTTTTTTTGAAGCCTTAAATCAAGATTAGTTGCT | 24 |
| Z3_A | CTCAAATATCAAACCCTCAATCAATATCTTTTTTTTTTGTGTCC | 25 |
| Z3_B | GGACACTTTTTTTTTTTTTGAAGCCTTAAATCAAGATTAGTTGCT | 26 |
| Z3_avi_A1 | GGACACTTTTTTTTTCTTGCGGGAGGTTTTGAA | 27 |
| Z3_avi_A2 | GGACACTTTTTTTTTCTTAAATCAAGATTAGTTGC | 28 |
| Z3_avi_B1 | CTCAAATATCAAACCCTCAATTTTTTTTTTGTGTCC | 29 |
| Z3_avi_B2 | CAATATCTGGTCAGTTGGCATTTTTTTTTGTGTCC | 30 |
| Z4_avi_A1 | GGACATTTTTTTTTTCTTGCGGGAGGTTTTGAA | 31 |
| Z4_avi_A2 | GGACATTTTTTTTTTCTTAAATCAAGATTAGTTGC | 32 |
| Z4_avi_B1 | CTCAAATATCAAACCCTCAATTTTTTTTTTATGTCC | 33 |
| Z4_avi_B2 | CAATATCTGGTCAGTTGGCATTTTTTTTTATGTCC | 34 |
| B1_Z1 | ATGGACAT | — |
| B1_Z1_7 | TGGACAT | — |
| B1_Z2 | TGGACAT | — |
| B1_Z3 | GGACAC | — |
| B1_Z4 | GGACAT | — |

### Results

To adapt fluorophores to the nanoswitch platform, two oligonucleotides were included onto the nanoswitch that are composed of a region complimentary to the nanoswitch, a flexible linker, and a short detection oligonucleotide region with an Amino dT C6 for fluorophore attachment. As shown in FIG. 2C, to adapt fluorophores to the nanoswitch platform, two oligonucleotides were included onto the nanoswitch that are composed of a region complimentary to the nanoswitch, a flexible linker, and a short FRET oligonucleotide region with an Amino dT C6 for fluorophore attachment. For a flexible linker, an 18-atom hexa-ethyleneglycol spacer was used. The two oligonucleotides are hybridized adjacent to and just inside where the target interactions are hybridized. One oligonucleotide is attached to Alexa 555 as a donor and the other oligonucleotide is attached to Alexa 647 as the acceptor for FRET. The short detection oligonucleotide regions at the ends of the linkers can bind to each other in a zipper configuration. The zipper is weak and only forms when the target interactions bind to each other to close the nanoswitch. As shown in FIG. 8A, fluorophore-labeled detection oligos are attached to the nanoswitch just inside two interacting targets. The nanoswitches close and the detection oligonucleotides hybridize to enable FRET only when the target interactions bind to each other. FIG. 8B shows multiple target interactions can be included to add an avidity effect to assay weakly two weakly interacting targets. The zipper serves two purposes, it positions the fluorophores relative to each other for maximum FRET efficiency and also enhances the affinity of weak target interactions.

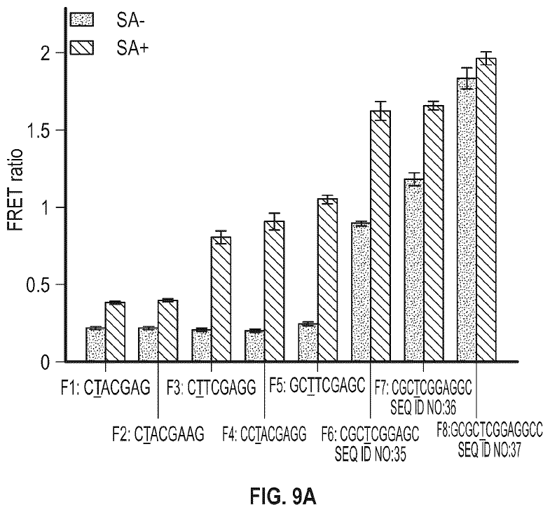

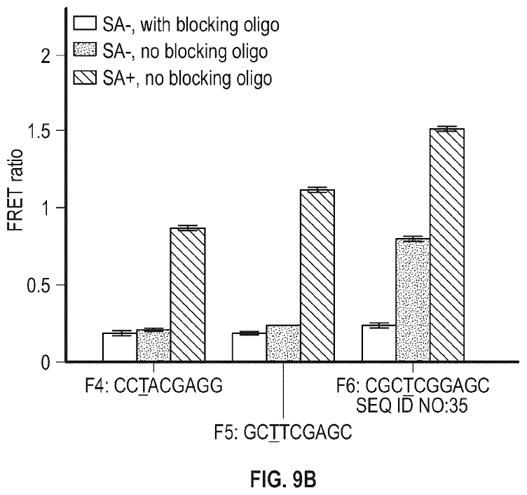

Next, the affinity of the detection oligonucleotides that maximized FRET without causing increased background was determined. To do this, nanoswitches were constructed with 2 biotin sites and 2 FRET detection oligos (FIG. 9A). The FRET oligonucleotides are adjacent to the biotin oligonucleotides, so are separated by ˜30 base pairs. The FRET oligos F1-F8 were designed to have increasing affinity. After hybridization, excess oligonucleotides were removed with a Blue Pippin. Then the nanoswitches were diluted to 1 nM and incubated for 1 hour in the absence or presence of streptavidin (SA), which can causes a loop to form between the 2 biotin sites. Then the FRET was measured from 7 μl samples in black 384-well plates using a BioTek Neo. It was found that detection oligos F4 and F5 yielded strong FRET in the presence of SA, without increasing background in the absence of SA. Indeed, additional samples including an oligonucleotide that binds to and blocks the detection oligonucleotides from forming a zipper do not further decrease signal for F4 or F5, but do decrease signal for F6 (FIG. 9B).

FIGS. 9A-9B show FRET as a function of FRET detection oligonucleotide affinity. Ratio of emission at 665 nm to 590 nm of 1 nM of biotin-modified nanoswitches for increasing FRET detection oligonucleotide affinity from V1 to V8. Results are given in the 0 nM SA (light grey) and 6 nM SA. Ratio of emission at 665 nm to 590 nm of 1 nM of biotin-modified nanoswitches for increasing detection oligonucleotide affinity from V4 to V6. Results are given in the presence of 50 μM short oligonucleotide that blocks the detection oligonucleotide, 0 nM SA (light grey) and 6 nM SA. The fluorophore-labeled base is underlined for each sequence. Error bars are SEM over 3 replicates.

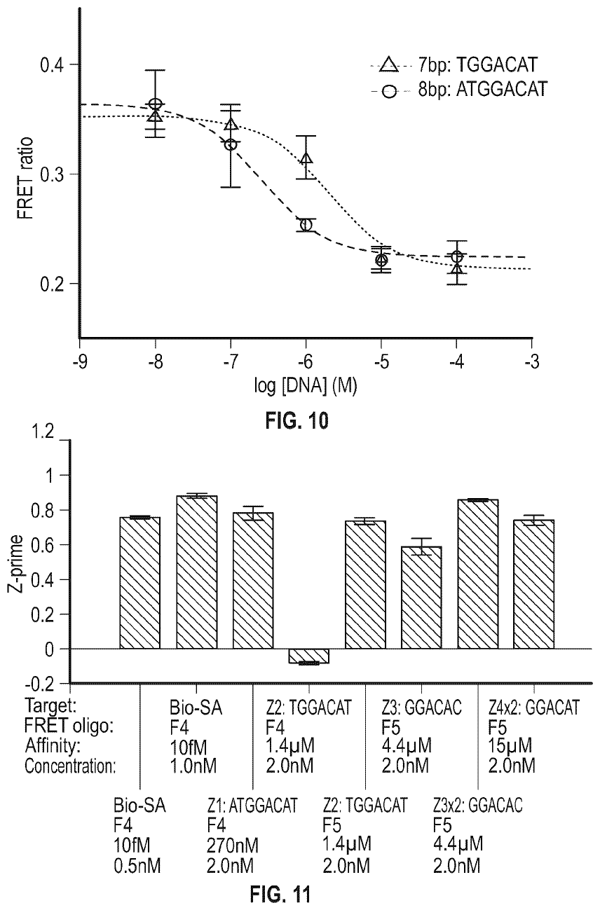

To demonstrate that the readout can be used to obtain dose response curves that can distinguish between ligands with different affinities, nanoswitches were created in which the target interaction consists of a short 8-mer DNA zipper (Z1: ATGTCCAT) with a 9T spacer from the nanoswitch attachment region. FRET was measured in the presence of increasing concentrations of 7-mer (TGGACAT) or 8-mer (ATGGACAT) blocking oligonucleotides that bind to one side of the zipper. FIG. 10 shows dose response curves for a target interaction sequence. The ratio of emission is shown at 665 nm to 590 nm of 1 nM of nanoswitches with a short target interaction sequence (Z1) in the presence of difference concentrations of short oligonucleotides that block this target interaction. Results are given for 7 bp (triangle, dotted-line) and 8 bp (circle, dashed-line) blocking oligonucleotides. The Ki for the 7 bp sequence is 2.0 μM (1.2 μM, 3.8 μM) and the Ki for the 8 bp sequence is 260 nM (150 nM, 430 nM), where the values in parenthesis are 95% confidence intervals using bootstrapping. Oligo sequences are given in Table 1. Error bars are SEM over 4 replicates. As expected, the 7-mer has a much higher Ki (2.0 μM) than the 8-mer Ki (260 nM), demonstrating that the system can be used to distinguish relative affinities.

Next the suitability of the assay for use in an HTS setting was evaluated by measuring the Z-prime (Z′) for strong and weak interactions. Shown in FIG. 11 are results of assay quality Z-prime using nanoswitch-FRET. DNA nanoswitches with target interactions of biotin-streptavidin or weak DNA-DNA zippers using either F4 or F5 for the detection oligonucleotides for concentrations ranging from 0.5 nM to 2.0 nM. For the biotin-SA target interaction, the background control is in the absence of SA while the signal control is in the presence of SA. For the weak DNA-DNA zipper target interactions, the background control is in the presence of 50 μM (or 250 μM for Z4) blocking oligonucleotide that binds to one part of the zipper, while the signal control is in the absence of any blocking oligonucleotide. The right-most two columns were performed using an avidity effect with 2 copies of each target (indicated by x2). Z′ was calculated as 1−3(σs+σb)/(μs−μb) for each plate over 5 different points for each condition and the error bars are SEM over 3 plates. Z′ is defined as 1−3(σs+σb)/(μs−μb), where σs is the standard deviation of the sample σb is the standard deviation of the background, μs is the average signal of the sample, and μb is the average signal of the background (Zhang et al. 1999). Z′ is a measure that combines the signal level above background and variability to quantify the statistical performance of an assay and assays with a Z′ of at least 0.5 are well-suited for HTS. First Z′ was measured for biotin nanoswitches that close in the presence of SA, an extremely strong interaction with a Kd of approximately 10 fM (Green 1975). With F4 as the FRET oligonucleotides and concentrations of 0.5 nM and 1.0 nM, it was found that Z′ reached 0.76 and 0.88, respectively. For a short oligonucleotide with predicted Kd of 270 nM, the Z′ also reaches a 0.78. For a short oligonucleotide with a high predicted Kd of 1.4 μM, though the Z′ drops to −0.8, but using a stronger affinity FRET oligo, F5, instead increases Z′ up to 0.73. The Z′ decreases to 0.59 when measuring an oligonucleotide with an even higher predicted Kd of 4.4 μM. An avidity effect was then included by hybridizing on 2 copies of the target oligonucleotides next to each FRET oligonucleotide. This enables us to reach Z′=0.86 for the same oligonucleotide with a predicted Kd of 4.4 μM and 0.74 for an oligonucleotide with a Kd of 15 μM.

### DISCUSSION

Demonstrated herein is nanoswitch-FRET for assaying modulation of weak molecular interactions in a format suitable for HTS. Using short complimentary DNA sequences Z′ scores of approximately 0.75 for interactions as weak as 15 μM were shown for nanoswitch-FRET.

### REFERENCES

Arkin M R, Tang Y, Wells J A. Small-molecule inhibitors of protein-protein interactions: progressing toward the reality. Chem Biol.2014; 21(9):1102-1114.
Bazin H, Preaudat M, Trinquet E, Mathis G. Homogeneous time resolved fluorescence resonance energy transfer using rare earth cryptates as a tool for probing molecular interactions in biology. Spectrochimica acta. Part A, Molecular and biomolecular spectroscopy 2001; 57(11):2197-211.
Eglen R M, Reisine T, Roby P, et al. The use of AlphaScreen technology in HTS: current status. Curr Chem Genomics. 2008; 1:2-10. Published 2008 Feb 25.
Fenwick C, Turelli P, Pellaton C, Farina A, Campos J, Raclot C, Pojer F, Cagno V, Nusslé S G, D′Acremont V, Fehr J, Puhan M, Pantaleo G, Trono D. A high-throughput cell- and virus-free assay shows reduced neutralization of SARS-CoV-2 variants by COVID-19 convalescent plasma. Sci Transl Med. 2021 Aug. 4; 13(605):eabi8452.
Green N M. Avidin. Advances in Protein Chemistry. 1975; 29: 85-133.
Hall M D, Yasgar A, Peryea T, et al. Fluorescence polarization assays in high-throughput screening and drug discovery: a review. Method Appl Fluoresc. 2016:4(2):022001.
Halvorsen K, Schaak D, Wong W P. Nanoengineering a single-molecule mechanical switch using DNA self-assembly. Nanotechnology. 2011;22(49):494005.
Hansen C H, Yang D, Koussa M A, Wong W P. Nanoswitch-linked immunosorbent assay (NLISA) for fast, sensitive, and specific protein detection. Proc Natl Acad Sci USA. 2017; 114(39):10367-10372.
Heyduk E, Dummit B, Chang Y H, Heyduk T. Molecular pincers: antibody-based homogeneous protein sensors. Anal Chem. 2008; 80(13):5152-5159.
Heyduk T, Heyduk E, Knoll E. Biosensors for detecting macromolecules and other analytes. U.S. Pat. No. 8,592,202B2.
Janzen, W P (Ed.). High Throughput Screening Methods and Protocols. New York City, New York: Springer; 2016.
Koussa M A, Halvorsen K, Ward A, Wong W P. DNA nanoswitches: a quantitative platform for gel-based biomolecular interaction analysis. Nat Methods. 2015; 12(2):123-126.
Krüttgen A, Lauen M, Klingel H, Imohl M, Kleines M. Two novel SARS-CoV-2 surrogate virus neutralization assays are suitable for assessing successful immunization with mRNA-1273. J Virol Methods. 2021 Sep. 23; 299:114297.
Mabonga L, Kappo A P. Protein-protein interaction modulators: advances, successes and remaining challenges. Biophys Rev. 2019; 11(4):559-581.
Milhas S, Raux B, Betzi S, Derviaux C, Roche P, Restouin A, Basse M J, Rebuffet E, Lugari A, Badol M, Kashyap R, Lissitzky J C, Eydoux C, Hamon V, Gourdel M E, Combes S, Zimmermann P, Aurrand-Lions M, Roux T, Rogers C, Müller S, Knapp S, Trinquet E, Collette Y, Guillemot J C, Morelli X. Protein-Protein Interaction Inhibition (2P2I)-Oriented Chemical Library Accelerates Hit Discovery. ACS chemical biology 2016; 11(8):2140-8.
Mravinacova S, Jönsson M, Christ W, Klingstrom J, Yousef J, Hellstrom C, Hedhammar M, Havervall S, Thålin C, Pin E, Tegel H, Nilsson P, Minberg A, Hober S. A cell-free high throughput assay for assessment of SARS-CoV-2 neutralizing antibodies. N Biotechnol. 2021 Oct. 7; 66:46-52.
Ramanathan M, Ferguson I D, Miao W, Khavari P A. SARS-CoV-2 B.1.1.7 and B.1.351 spike variants bind human ACE2 with increased affinity. Lancet Infect Dis. 2021 Aug;21(8):1070.
Scott D E, Bayly A R, Abell C, Skidmore J. Small molecules, big targets: drug discovery faces the protein-protein interaction challenge. Nat Rev Drug Discov. 2016 August; 15(8):533-50.
Tan C W, Chia W N, Qin X, Liu P, Chen M L, Tiu C, Hu Z, Chen V C, Young B E, Sia W R, Tan Y J, Foo R, Yi Y, Lye D C, Anderson D E, Wang L F. A SARS-CoV-2 surrogate virus neutralization test based on antibody-mediated blockage of ACE2-spike protein-protein interaction. Nat Biotechnol. 2020 September;38(9):1073-1078.
Wigle T J, Herold J M, Senisterra G A, Vedadi M, Kireev D B, Arrowsmith C H, Frye S V, Janzen W P. Screening for inhibitors of low-affinity epigenetic peptide-protein interactions: an AlphaScreen-based assay for antagonists of methyl-lysine binding proteins. Journal of biomolecular screening 2010; 15(1):62-71.
Zhang J H, Chung T D, Oldenburg K R. A Simple Statistical Parameter for Use in Evaluation and Validation of High Throughput Screening Assays. Journal of biomolecular screening. 1999; 4(2):67-73.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.
In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system for analyzing modulation of a binding interaction, comprising
a first member of a binding pair, conjugated to a first oligonucleotide, wherein the first oligonucleotide is conjugated to a FRET donor fluorophore, and
a second member of the binding pair, conjugated to a second oligonucleotide, wherein the second oligonucleotide is conjugated to a FRET acceptor fluorophore,
wherein the first and second members bind to each other with a member dissociation constant, Kd, in the range of about 100 pM to about 100 μM, and wherein the first and second oligonucleotides hybridize to each other with an oligonucleotide dissociation constant, Kd, that ranges from about 100 pM to about 1 μM.

2. The system of claim 1, wherein the first member is conjugated to the first oligonucleotide using a first linker.

3. The system of claim 1 or 2, wherein the second member is conjugated to the second oligonucleotide using a second linker.

4. The system of claim 2 or 3, wherein the first and/or the second linker has a length of about 2-14 nm.

5. The system of claim 2 or 3, wherein the first and/or the second linker comprises 1-6 adjacent 18-atom hexa-ethyleneglycol spacers.

6. The system of claim 2 or 3, wherein the first and/or the second linker comprises four adjacent 18-atom hexa-ethyleneglycol spacers.

7. The system of any of the foregoing claims, wherein the first member of the binding pair is a virus or viral component capable of binding to a mammalian cell and the second member of the binding pair is a mammalian cell or a component of the mammalian cell to which the viral component binds.

8. The system of claim 7, wherein the virus is Sars-CoV-2 or the viral component is Sars-CoV-2 receptor binding domain (RBD) and the mammalian cell is a human cell or the component of the mammalian cells is receptor ACE2.

9. The system of claim 7 or 8, wherein the member Kd is about 400 nM and the oligonucleotide Kd is about 50 nM.

10. The system of any one of the foregoing claims, wherein once the first and second oligonucleotides are hybridized to each other, the donor and acceptor FRET fluorophores are spaced apart by 1 to 8 base pairs, optionally 4-7 base pairs, further optionally 4 or 5 base pairs.

11. The system of any one of the foregoing claims, wherein the donor FRET fluorophore is Alexa 555 and the acceptor FRET fluorophore is Alexa 647, or wherein the donor FRET fluorophore is Alexa 488 and the acceptor FRET fluorophore is Alexa 555, or wherein the donor FRET fluorophore is Alexa 594 and the acceptor FRET fluorophore is Alexa 647.

12. The system of any one of the foregoing claims, wherein the first member and/or second member is a protein.

13. A method for detecting an agent capable of modulating a binding interaction, comprising
(a) providing (i) a first member of a binding pair, conjugated to a first oligonucleotide, wherein the first oligonucleotide is conjugated to a FRET donor fluorophore, and (ii) a second member of the binding pair, conjugated to a second oligonucleotide, wherein the second oligonucleotide is conjugated to a FRET acceptor fluorophore, wherein the first and second members bind to each other with a member dissociation constant, Kd, in the range of about 100 pM nM to about 100 μM, and wherein the first and second oligonucleotides hybridize to each other with an oligonucleotide dissociation constant, Kd, that ranges from about 100 pM to about 1 μM and is comparable to or greater than the concentration of oligonucleotides used in the assay,
(b) combining the first member and the second member with a sample, under conditions that allow binding of the first member to the second member and that allow binding of the first oligonucleotide to the second oligonucleotide, optionally wherein the first member and second member are provided in concentrations ranging from about 100 pM to about 100 nM,
(c) measuring test fluorescence from the FRET acceptor, and
(d) (1) identifying the sample as containing an agent that
(i) decreases binding of the first member to the second member as a test fluorescence that is less than a control fluorescence measured after combining the first member and the second member under the same conditions as (b) but in the absence of the sample, or
(ii) increases binding of the first member to the second member as a test fluorescence that is greater than a control fluorescence measured after combining the first member and the second member under the same conditions as (b) but in the absence of the sample; or
(2) identifying the sample as containing an agent that modulates binding of the first member to the second member, wherein a test fluorescence that is less than a control fluorescence, measured in the absence of the sample, is indicative of an agent that decreases binding of the first member to the second member, and a test fluorescence that is greater than a control fluorescence, measured in the absence of the sample, is indicative of an agent that increases binding of the first member to the second member.

14. The method of claim 13, wherein the oligonucleotide Kd is about 1 times, 2 times, 5 times, 10 times or higher than the concentration of oligonucleotides used in the assay.

15. The method of claim 13 or 14, wherein the first member is conjugated to the first oligonucleotide using a first linker.

16. The method of any one of claims 13-15, wherein the second member is conjugated to the second oligonucleotide using a second linker.

17. The method of claim 15 or 16, wherein the first and/or the second linker has a length of about 2-14 nm.

18. The method of claim 15 or 16, wherein the first and/or the second linker comprises 1-6 adjacent 18-atom hexa-ethyleneglycol spacers.

19. The method of claim 15 or 16, wherein the first and/or the second linker comprises four adjacent 18-atom hexa-ethyleneglycol spacers.

20. The method of any one of claims 13-19, wherein the method is a method that detects an agent that decreases binding of the first member to the second member.

21. The method of any one of claims 13-19, wherein the method is a method that detects an agent that increases binding of the first member to the second member.

22. The method of any one of claims 13-21, wherein the sample is a blood or serum sample, optionally a blood or serum sample from a human subject, further optionally a human serum sample.

23. The method of any one of claims 13-22, wherein the first member of the binding pair is a virus or viral component capable of binding to a mammalian cell and the second member of the binding pair is a mammalian cell or a component of the mammalian cell to which the viral component binds.

24. The method of claim 23, wherein the virus is Sars-CoV-2 or the viral component is Sars-CoV-2 receptor binding domain (RBD) and the mammalian cell is a human cell or the component of the mammalian cells is receptor ACE2.

25. The method of claim 23 or 24, wherein the member Kd is about 400 nM and the oligonucleotide Kd is about 50 nM.

26. The method of any one of claims 23-25, wherein the method is a method that detects an agent that decreases binding of the first member to the second member and the agent is an antibody, and the sample is a blood or serum sample.

27. The method of any one of claims 13-26, wherein once the first and second oligonucleotides are hybridized to each other, the donor and acceptor FRET fluorophores are spaced apart by 1 to 8 base pairs, optionally 4-7 base pairs, further optionally 4 or 5 base pairs.

28. The method of any one of claims 13-27, wherein the donor FRET fluorophore is Alexa 555 and the acceptor FRET fluorophore is Alexa 647, or wherein the donor FRET fluorophore is Alexa 488 and the acceptor FRET fluorophore is Alexa 555, or wherein the donor FRET fluorophore is Alexa 594 and the acceptor FRET fluorophore is Alexa 647.

29. The method of any one of claims 13-28, wherein the first and second members are provided at concentrations lower than the member Kd, optionally 10-fold lower, 50-fold lower, 100-fold lower, 500-fold lower, 1,000-fold lower, or 5,000 lower.

30. The method of any one of claims 13-29, wherein the first member and second member and sample are combined at about room temperature for about 30 minutes.

31. The method of any one of claims 13-30, wherein the method is carried out in a single well of a multiwell plate, optionally wherein the multiwell plate is a 384-well plate.

32. The method of any one of claims 13-31, wherein the first member and/or second member is a protein.

33. The method of any one of claims 13-32, wherein the method does not comprise a washing step between steps (b) and (c).

34. The method of any one of claims 13-33, wherein the first and second member are in free-form in solution.

35. The method of any one of claims 13-34, wherein the first and second members are attached to a single polymer, with sufficient distance between the first and second members to allow binding of the first and second members to each other.

36. A system for analyzing modulation of a binding interaction, comprising
a first member of a binding pair, conjugated to a first oligonucleotide, wherein the first oligonucleotide is conjugated to a fluorophore, and
a second member of the binding pair, conjugated to a second oligonucleotide,
wherein the first and second members bind to each other with a member dissociation constant, Kd, in the range of about 100 pM nM to 100 μM and wherein the first and second oligonucleotides hybridize to each other with an oligonucleotide dissociation constant, Kd, that ranges from about 100 pM to about 1 μM.

37. The system of claim 36, wherein the first member is conjugated to the first oligonucleotide using a linker.

38. The system of claim 37, wherein the linker has a length of about 2-14 nm.

39. The system of claim 37, wherein the linker comprises 1-6 adjacent 18-atom hexa-ethyleneglycol spacers.

40. The system of claim 37, wherein the first linker comprises four adjacent 18-atom hexa-ethyleneglycol spacers.

41. A method for detecting an agent capable of modulating a binding interaction, comprising
(a) providing (i) a first member of a binding pair, conjugated to a first oligonucleotide, wherein the first oligonucleotide is conjugated to a fluorophore, and (ii) a second member of the binding pair, conjugated to a second oligonucleotide, wherein the first and second members bind to each other with a member dissociation constant, Kd, in the range of about 100 pM to about 100 μM, and wherein the first and second oligonucleotides hybridize to each other with an oligonucleotide dissociation constant, Kd, that ranges from about 100 pM to about 1 μM and is about equal to or greater than the concentration of oligonucleotides used in the assay, optionally wherein said concentrations are about 100 pM to about 100 nM,
(b) combining the first member and the second member with a sample, under conditions that allow binding of the first member to the second member and that allow binding of the first oligonucleotide to the second oligonucleotide,
(c) measuring test fluorescence using fluorescence polarization, and
(d) identifying the sample as containing an agent that
(i) decreases binding of the first member to the second member as a test fluorescence that is less than a control fluorescence measured after combining the first member and the second member under the same conditions as (b) but in the absence of the sample, or
(ii) increases binding of the first member to the second member as a test fluorescence that is greater than a control fluorescence measured after combining the first member and the second member under the same conditions as (b) but in the absence of the sample.

42. A system for analyzing modulation of a binding interaction, comprising a polymer conjugated to
(i) a first member of a binding pair and a second member of a binding pair, wherein the first and second members bind to each other with a member dissociation constant, Kd, of 100 pM to 100 μM, and wherein the polymer adopts a looped conformation when the first and second member bind to each other, and
(ii) a first FRET oligonucleotide conjugated to a first FRET fluorophore and a second FRET oligonucleotide conjugated to a second FRET fluorophore, wherein the first and second FRET oligonucleotides hybridize to each other with a FRET oligonucleotide dissociation constant, Kd, and wherein one of the FRET fluorophores is a FRET donor fluorophore and the other of the FRET fluorophores is a FRET acceptor fluorophore.

43. The system of claim 42, wherein the polymer is a nucleic acid, a linear amino acid chain, or polyethylene glycol.

44. A system for analyzing modulation of a binding interaction, comprising
a complex comprising a single-stranded scaffold nucleic acid hybridized to a plurality of single-stranded oligonucleotides thereby forming a linear partially double-stranded nucleic acid,
wherein a first single-stranded oligonucleotide in the plurality is linked to a first member of a binding pair, and a second single-stranded oligonucleotide in the plurality is linked to a second member of a binding pair, wherein the first and second members bind to each other with a member dissociation constant, Kd, of 100 pM to 100 μM, and wherein the complex adopts a looped conformation when the first and second member bind to each other,
wherein a first FRET oligonucleotide conjugated to a first FRET fluorophore is conjugated to the complex and a second FRET oligonucleotide conjugated to a second FRET fluorophore is conjugated to the complex, wherein the first and second FRET oligonucleotides hybridize to each other with a FRET oligonucleotide dissociation constant, Kd, optionally in the range of about 100 pM to 1 μM, and wherein one of the FRET fluorophores is a FRET donor fluorophore and the other of the FRET fluorophores is a FRET acceptor fluorophore.

45. The system of claim 44, wherein the first FRET oligonucleotide is conjugated to the complex using a first linker and/or the second FRET oligonucleotide is conjugated to the complex using a second linker, optionally wherein the first and/or second linker comprises one or more 18-atom hexa-ethyleneglycol spacers.

46. The system of claim 44, wherein the oligonucleotides are positioned along the length of the complex in order, optionally as first oligonucleotide, first FRET oligonucleotide, second FRET oligonucleotide, and second oligonucleotide.

47. The system of any one of claims 44-46, wherein the first and second FRET oligonucleotides bind to each other only upon binding of the first and second members to each other.

48. The system of any one of claims 44-47, wherein the first oligonucleotide and the first FRET oligonucleotide are about 30 base pairs apart.

49. The system of any one of claims 44-48, wherein the second oligonucleotide and the second FRET oligonucleotide are about 30 base pairs apart.

50. The system of any one of claims 44-49, wherein a third and a fourth oligonucleotide, flank the first and second oligonucleotides respectively, comprise a first member and a second member respectively.

51. A method for detecting an agent capable of modulating a binding interaction, comprising
(a) combining the polymer of claim 42 or 43 with a sample, under conditions that allow binding of the first member to the second member and that allow binding of the first FRET oligonucleotide to the second FRET oligonucleotide,
(b) measuring test fluorescence from the FRET acceptor, and
(c) identifying the sample as containing an agent that
(i) decreases binding of the first member to the second member as a test fluorescence that is less than a control fluorescence measured after incubating the polymer under the same conditions as (a) in the absence of the sample, or
(ii) increases binding of the first member to the second member as a test fluorescence that is greater than a control fluorescence measured after incubating the polymer under the same conditions as (a) in the absence of the sample.

52. A method for detecting an agent capable of modulating a binding interaction, comprising
(a) combining the complex of any one of claims 44-50 with a sample, under conditions that allow binding of the first member to the second member and that allow binding of the first FRET oligonucleotide to the second FRET oligonucleotide,
(b) measuring test fluorescence from the FRET acceptor, and
(c) identifying the sample as containing an agent that
(i) decreases binding of the first member to the second member as a test fluorescence that is less than a control fluorescence measured after incubating the complex under the same conditions as (a) in the absence of the sample, or
(ii) increases binding of the first member to the second member as a test fluorescence that is greater than a control fluorescence measured after incubating the complex under the same conditions as (a) in the absence of the sample.


SEQUENCE LISTING (37 sequences)

SEQ ID NO: 1 - DNA, 30 bp, Artificial Sequence
  Synthetic
tcagttggcaaatcaacagttgaaaggaat

SEQ ID NO: 2 - DNA, 29 bp, Artificial Sequence
  Synthetic
gttttagcgaacctcccgacttgcgggag

SEQ ID NO: 3 - DNA, 30 bp, Artificial Sequence; identical to SEQ ID NO: 1
  Synthetic

SEQ ID NO: 4 - DNA, 29 bp, Artificial Sequence
  Synthetic
gttttagcgaacctcccgacttgcgggag

SEQ ID NO: 5 - DNA, 30 bp, Artificial Sequence; identical to SEQ ID NO: 1
  Synthetic

SEQ ID NO: 6 - DNA, 29 bp, Artificial Sequence
  Synthetic
gttttagcgaacctcccgacttgcgggag

SEQ ID NO: 7 - DNA, 30 bp, Artificial Sequence; identical to SEQ ID NO: 1
  Synthetic

SEQ ID NO: 8 - DNA, 29 bp, Artificial Sequence
  Synthetic
gttttagcgaacctcccgacttgcgggag

SEQ ID NO: 9 - DNA, 30 bp, Artificial Sequence; identical to SEQ ID NO: 1
  Synthetic

SEQ ID NO: 10 - DNA, 29 bp, Artificial Sequence
  Synthetic
gttttagcgaacctcccgacttgcgggag

SEQ ID NO: 11 - DNA, 30 bp, Artificial Sequence; identical to SEQ ID NO: 1
  Synthetic

SEQ ID NO: 12 - DNA, 29 bp, Artificial Sequence
  Synthetic
gttttagcgaacctcccgacttgcgggag

SEQ ID NO: 13 - DNA, 30 bp, Artificial Sequence; identical to SEQ ID NO: 1
  Synthetic

SEQ ID NO: 14 - DNA, 29 bp, Artificial Sequence
  Synthetic
gttttagcgaacctcccgacttgcgggag

SEQ ID NO: 15 - DNA, 30 bp, Artificial Sequence; identical to SEQ ID NO: 1
  Synthetic

SEQ ID NO: 16 - DNA, 29 bp, Artificial Sequence
  Synthetic
gttttagcgaacctcccgacttgcgggag

SEQ ID NO: 17 - DNA, 20 bp, Artificial Sequence
  Synthetic
aatcaacagttgaaaggaat

SEQ ID NO: 18 - DNA, 18 bp, Artificial Sequence
  Synthetic
gttttagcgaacctcccg

SEQ ID NO: 19 - DNA, 29 bp, Artificial Sequence
  Synthetic
ctcaaatatcaaaccctcaatcaatatct

SEQ ID NO: 20 - DNA, 30 bp, Artificial Sequence
  Synthetic
ttttgaagccttaaatcaagattagttgct

SEQ ID NO: 21 - DNA, 43 bp, Artificial Sequence
  Synthetic
ctcaaatatcaaaccctcaatcaatatctttttttatgtccat

SEQ ID NO: 22 - DNA, 44 bp, Artificial Sequence
  Synthetic
atggacatttttttttttgaagccttaaatcaagattagttgct

SEQ ID NO: 23 - DNA, 45 bp, Artificial Sequence
  Synthetic
ctcaaatatcaaaccctcaatcaatatcttttttttttatgtcca

SEQ ID NO: 24 - DNA, 46 bp, Artificial Sequence
  Synthetic
tggacattttttttttttttgaagccttaaatcaagattagttgct

SEQ ID NO: 25 - DNA, 44 bp, Artificial Sequence
  Synthetic
ctcaaatatcaaaccctcaatcaatatcttttttttttgtgtcc

SEQ ID NO: 26 - DNA, 45 bp, Artificial Sequence
  Synthetic
ggacactttttttttttttgaagccttaaatcaagattagttgct

SEQ ID NO: 27 - DNA, 33 bp, Artificial Sequence
  Synthetic
ggacactttttttttcttgcgggaggttttgaa

SEQ ID NO: 28 - DNA, 35 bp, Artificial Sequence
  Synthetic
ggacactttttttttcttaaatcaagattagttgc

SEQ ID NO: 29 - DNA, 36 bp, Artificial Sequence
  Synthetic
ctcaaatatcaaaccctcaattttttttttgtgtcc

SEQ ID NO: 30 - DNA, 35 bp, Artificial Sequence
  Synthetic
caatatctggtcagttggcatttttttttgtgtcc

SEQ ID NO: 31 - DNA, 33 bp, Artificial Sequence
  Synthetic
ggacattttttttttcttgcgggaggttttgaa

SEQ ID NO: 32 - DNA, 35 bp, Artificial Sequence
  Synthetic
ggacattttttttttcttaaatcaagattagttgc

SEQ ID NO: 33 - DNA, 36 bp, Artificial Sequence
  Synthetic
ctcaaatatcaaaccctcaattttttttttatgtcc

SEQ ID NO: 34 - DNA, 35 bp, Artificial Sequence
  Synthetic
caatatctggtcagttggcatttttttttatgtcc

SEQ ID NO: 35 - DNA, 10 bp, Artificial Sequence
  Synthetic
cgctcggagc

SEQ ID NO: 36 - DNA, 11 bp, Artificial Sequence
  Synthetic
cgctcggaggc

SEQ ID NO: 37 - DNA, 13 bp, Artificial Sequence
  Synthetic
gcgctcggaggcc